(19) United States
(12) Patent Application Publication
(10) Pub. No.: US 2021/0292370 A1
(43) Pub. Date: Sep. 23, 2021

(54) HUMAN ANTI-AAV2 CAPSID POLYCLONAL ANTIBODY EPITOPES

(71) Applicant: OREGON HEALTH & SCIENCE UNIVERSITY, Portland, OR (US)

(72) Inventors: Kei Adachi, Portland, OR (US); Xiao Lan Chang, Portland, OR (US); Hiroyuki Nakai, Portland, OR (US)

(21) Appl. No.: 17/088,977

(22) Filed: Nov. 4, 2020

Related U.S. Application Data
(63) Continuation of application No. PCT/US2019/030955, filed on May 6, 2019.
(60) Provisional application No. 62/667,360, filed on May 4, 2018.

(51) Int. Cl. C07K 14/005; C12N 15/10

(52) U.S. Cl. CPC C07K 14/005; C12N 2750/14122; C12N 15/1065

(57) ABSTRACT

Human anti-AAV capsid polyclonal antibody conformational epitopes including those of neutralizing antibodies are provided. The epitopes can be recognized by human anti-AAV2 or other AAV strain-derived capsid polyclonal antibodies. One or more of the epitopes may be mutated to form AAV2 and other AAV strain-derived capsids that can escape antibody neutralization. Methods of identifying human anti-AAV capsid polyclonal antibody conformational epitopes are also provided.

### RELATED APPLICATIONS

This application is a continuation of International Patent Application PCT/US19/30955, which was filed on May 6, 2019, which in turn claims priority to U.S. Provisional Patent Application No. 62/667,360, which was filed on May 4, 2018, both of which are hereby incorporated by reference in their entirety.

### STATEMENT REGARDING FEDERALLY SPONSORED RESEARCH

This invention was made with government support under Grant No. NS088399 awarded by the National Institutes of Health. The government has certain rights in the invention.

### BACKGROUND

Viral neutralizing antibody (NtAb) epitope mapping can assist in the development of new vaccines and pharmaceuticals for the prevention and/or treatment of infectious diseases. Additionally, viral NtAb epitope mapping can assist in the development of gene delivery vectors. Identification of and knowledge regarding viral NtAb epitopes may help in the genetic engineering of components of viral vectors that can evade a host immune response, as the host immune response can be an obstacle to effective in vivo gene therapy.

Adeno-associated virus (AAV) is a promising in vivo gene delivery vector for gene therapy. Various issues remain to be overcome, however, in the use of AAV as an in vivo gene delivery vector, including the need of a high vector dose for clinically beneficial outcomes, efficacy-limiting host immune response against viral proteins, promiscuous viral tropism, and the prevalence of pre-existing anti-AAV neutralizing antibodies in humans.

A number of naturally occurring serotypes and subtypes have been isolated from human and non-human primate tissues (Gao G et al., J Virol 78, 6381-6388 (2004) and Gao G et al., Proc Natl Acad Sci USA 99, 11854-11859 (2002)). Among the newly-identified AAV isolates, AAV serotype 8 (AAV8) and AAV serotype 9 (AAV9) have gained attention because recombinant AAV vectors derived from these two serotypes can transduce various organs including the liver, heart, skeletal muscles, and central nervous system with high efficiency following systemic administration via the periphery (Foust K D et al., Nat Biotechnol 27, 59-65 (2009); Gao et al., 2004, supra; Ghosh A et al., Mol Ther 15, 750-755 (2007); Inagaki K et al., Mol Ther 14, 45-53 (2006); Nakai H et al., J Virol 79, 214-224 (2005); Pacak C A et al., Circ Res 99, e3-e9 (2006); Wang Z et al., Nat Biotechnol 23, 321-328 (2005); and Zhu T et al., Circulation 112, 2650-2659 (2005)).

The robust transduction by AAV8 and AAV9 vectors has been presumed to be ascribed to strong tropism for these cell types, efficient cellular uptake of vectors, and/or rapid uncoating of virion shells in cells (Thomas C E et al., J Virol 78, 3110-3122 (2004)). In addition, emergence of capsid-engineered AAV vectors with better performance has significantly broadened the utility of AAV vectors as a vector toolkit (Asokan A et al., Mol Ther 20, 699-708 (2012)). Proof-of-concept for AAV vector-mediated gene therapy has been shown in many preclinical animal models of human diseases. Phase I/II clinical studies have been initiated or completed for genetic diseases including hemophilia B (Manno C S et al., Nat Med 12, 342-347 (2006) and Nathwani A C et al., N Engl J Med 365, 2357-2365 (2011)); muscular dystrophy (Mendell J R et al., N Engl J Med 363, 1429-1437 (2011)); cardiac failure (Jessup Met al., Circulation 124, 304-313 (2011)); blinding retinopathy (Maguire A M et al., Lancet 374, 1597-1605 (2009)); al anti-trypsin deficiency (Flotte T R et al., Hum Gene Ther 22, 1239-1247 (2011)); and spinal muscular atrophy (Mendell J R et al., N Engl J Med 377:1713-1722 (2017)); among others.

Although AAV vectors have widely been used in preclinical animal studies and have been tested in clinical safety studies, the current AAV vector-mediated gene delivery systems generally remain suboptimal for broader clinical applications. The sequence of an AAV viral capsid protein defines numerous features of a particular AAV vector. For example, the capsid protein affects features such as capsid structure and assembly, interactions with AAV nonstructural proteins such as Rep and AAP proteins, interactions with host body fluids and extracellular matrix, clearance of the virus from the blood, vascular permeability, antigenicity, reactivity to NtAbs, tissue/organ/cell type tropism, efficiency of cell attachment and internalization, intracellular trafficking routes, and virion uncoating rates. Furthermore, the relationship between a given AAV capsid amino acid sequence and the characteristics of the AAV vector are unpredictable.

High prevalence of pre-existing NtAbs against AAV capsids in humans poses a significant barrier to successful AAV vector-mediated gene therapy. There has been interest in developing “stealth” AAV vectors that can evade NtAbs; however, creation of such AAV vectors generally relies on more comprehensive information about NtAb epitopes, which currently remains limited as there is no method of easily and effectively mapping epitopes for polyclonal anti-AAV capsid antibodies present in animal and human sera.

DNA-barcoded AAV2R585E hexapeptide (HP) scanning capsid mutant libraries have been produced in which AAV2-derived HPs were replaced with those derived from other serotypes. These libraries have been injected intravenously into mice harboring anti-AAV1 or AAV9 capsid antibodies, which has led to the identification of 452-QSGSAQ-457 (SEQ ID NO:1) in the AAV1 capsid and 453-GSGQN-457 (SEQ ID NO:2) in the AAV9 capsid as epitopes for anti-AAV NtAbs in mouse sera (Adachi K et al., Nat Commun 5, 3075 (2014)). These epitopes correspond to the highest peak of the three-fold symmetry axis protrusion on the capsid. In addition, this region may also function as an epitope for mouse anti-AAV7 NtAbs using the same in vivo approach. A sequencing-based high-throughput approach, termed AAV Barcode-Seq, can allow characterization of phenotypes of hundreds of different AAV strains and can be applied to anti-AAV NtAb epitope mapping.

### BRIEF DESCRIPTION OF THE FIGURES

The embodiments disclosed herein will become more fully apparent from the following description and appended claims, taken in conjunction with the accompanying drawings. These drawings depict only typical embodiments, which will be described with additional specificity and detail through use of the accompanying drawings in which:

FIG. 1A depicts a map of a DNA-barcoded AAV genome containing a pair of 12 nucleotide-long DNA barcodes (It-VBC and rt-VBC) downstream of the AAV2 pA. Each virus barcode (VBC) can be PCR-amplified separately. This type of DNA-barcoded AAV vector genome was used in the inventors' earlier studies e.g., those published in Adachi K et al., Nat Commun 5:3075 (2014).

FIG. 1B depicts a map of a DNA-barcoded double-stranded (ds) AAV-U6-VBCLib vector genome. The dsAAV-U6-VBCLib vector genome harbors a human U6 small nuclear (sn) RNA promoter-driven nonfunctional noncoding RNA expression cassette of 0.6 kb containing a pair of 12 nucleotide-long DNA barcodes (It-VBC and rt-VBC) (Earley L F et al., J Virol 91 (2017).). The vector genome also contains stuffer DNA derived from the bacterial lacZ gene open reading frame (ORF). The dsAAV-U6-VBCLib vector plasm ids were designed to express DNA barcodes as RNA barcodes in AAV vector transduced cells (Adachi K et al., Mol Ther 22 (2014)). Many of our recent AAV Barcode-Seq studies including those presented below have used the dsAAV-U6-VBCLib AAV vector genome.

FIG. 1C is a representation of double alanine (AA) scanning mutagenesis of the AAV9 capsid.

FIG. 1D is a representation of hexapeptide (HP) scanning mutagenesis of the AAV2R585E capsid at a two amino acid interval.

FIG. 1E is a representation of a procedure for AAV Barcode-Seq analysis. PCR products obtained from each sample are indexed with sample-specific barcodes attached to the PCR primers. This allows multiplexed ILLUMINA sequencing. Phenotypic Difference (PD) values provide information about a spectrum of phenotypes (receptor binding, transduction, tropism, blood clearance, reactivity to NtAbs, blood-cerebrospinal fluid barrier (BCSFB) penetrability, etc.) for each serotype or mutant.

FIG. 2 depicts AAV9 hexapeptide (HP) scanning mutants and AAV9 dodecapeptide (DP) scanning mutants in which AAV9-derived HPs or DPs are replaced with those derived from AAV2.

FIG. 3 depicts AAV5 dodecapeptide (DP) scanning mutants in which AAV5-derived DPs are replaced with those derived from AAV2.

FIG. 4. depicts the procedure of IP-Seq.

FIG. 5 shows the results of mapping of conformational epitopes of polyclonal anti-AAV2 antibodies present in human serums samples using an AAV9-HP library. The data generated using four different cut-off values are compared. This analysis clearly reveals common human anti-AAV2 capsid polyclonal antibody conformational epitopes.

FIG. 6 depicts common human anti-AAV2 capsid polyclonal antibody conformational epitopes identified by IP-Seq with a library containing both AAV9-HP and AAV9-DP mutants.

FIG. 7 depicts common human anti-AAV2 capsid polyclonal antibody conformational epitopes identified by IP-Seq with an AAV5-DP mutant library.

FIG. 8 depicts common human anti-AAV2 capsid polyclonal neutralizing antibody conformational epitopes identified by in vivo PK-Seq with an AAV9-HP mutant library.

FIG. 9 depicts common human anti-AAV2 capsid polyclonal neutralizing antibody conformational epitopes identified by in vivo PK-Seq with an AAV9-DP mutant library.

FIG. 10 shows the method used to identify human anti-AAV capsid polyclonal neutralizing antibody-escaping mutants.

FIG. 11 shows the ability for AAV2, AAV2Ep123mt1 and AAV9 vectors to transduce CHO-K1 cells in the presence or absence of Gammagard, Immune Globulin Intravenous (Human), containing high titers of human anti-AAV capsid polyclonal neutralizing antibodies.

FIG. 12 shows the ELISA data showing that pre-incubation of 20 μl of serum samples or IVIG with 1×10¹¹ vg of AAV9 particles is sufficient to clear antibodies that bind AAV9 while preserving antibodies that bind AAV2.

FIGS. 13A-D collectively show the epitopes we have identified for AAV2 capsid and potential epitopes of other AAV serotypes (AAV1, 3B, 4, 5, 6, 7, 8, 9, 10, 11, 12 and 13).

FIG. 13A shows sequence alignment of AAV2 and other AAV serotypes (AAV1, 3B, 4, 5, 6, 7, 8, 9, 10, 11, 12 and 13).

FIG. 13B shows sequence alignment of AAV2 and other AAV serotypes (AAV1, 3b, 4, 5, 6, 7, 8, 9, 10, 11, 12 and 13) following the sequences in FIG. 13A.

FIG. 13C shows sequence alignemtn of AAV2 and other AAV serotypes (AAV1, 3B, 4, 5, 6, 7, 8, 9, 10, 11, 12 and 13) following the sequences in FIG. 13B.

FIG. 13D shows sequence alignment of AAV2 and other AAV serotypes (AAV1, 3B, 4, 5, 6, 7, 8, 9, 10, 11, 12 and 13) following the sequences in FIG. 13C.

FIG. 14 is an example showing that IP-Seq using AAV5-DP mutants can identify more amino acid sequences comprising an anti-AAV2 antibody that IP-Seq using AAV9-HP mutants cannot identify.

### DETAILED DESCRIPTION

It will be readily understood that the embodiments, as generally described herein, are exemplary. The following more detailed description of various embodiments is not intended to limit the scope of the present disclosure, but is merely representative of various embodiments. Moreover, the order of steps or actions of the methods disclosed herein may be changed by those skilled in the art without departing from the scope of the present disclosure. In other words, unless a specific order of steps or actions is required for proper operation of the embodiment, the order or use of specific steps or actions may be modified.

The present disclosure provides methods of identifying a mutant AAV capsid protein. In certain embodiments, the AAV capsid protein is “mutated” or “altered” with respect to the wild-type sequence of a first AAV strain, AAVx, wherein the mutant AAVx capsid protein comprises at least one altered capsid epitope, the method comprising the steps of (1) preparing a plurality of AAVx capsid mutants, wherein each AAVx capsid mutant comprises one or more altered amino acids and wherein each AAVx capsid mutant is indexed with a virus-specific barcode; (2) reacting the plurality of AAVx capsid mutants with a plurality of antibodies, wherein each antibody binds to one or more epitopes on an AAV capsid protein; (3) collecting the AAVx capsid mutants that bind to one or more antibodies; and (4) identifying the AAVx capsid mutants that bind to one or more antibodies. The mutant AAV capsid may be configured to escape antibody binding or neutralization.

When referring to a gene, the term “wild-type” is used in its ordinary sense and is defined as a gene that has the same protein-coding nucleotide sequence as the corresponding gene in an animal species, cell, or viral strain. For gene sequences that are polymorphic, “wild-type” refers to the sequence of the most common form of the gene in that animal species, cell, or viral strain. The term “wild-type” may also be used in connection with a protein whose amino acid sequence is identical to the most common form of that protein's amino acid sequence. When used in connection with a particular strain, e.g., an AAV strain, “wild-type” refers to the most common amino acid sequence of a particular protein in that strain. The terms “mutant” or “mutated” are also used in their ordinary sense and are defined as a gene that does not have the same protein-coding nucleotide sequence as the corresponding wild-type gene in that animal species, cell, or viral strain. A mutation may be one or more of (1) a change in one or more nucleotides, especially where such change alters the amino acid sequence encoded by the nucleotide sequence; (2) a deletion of one or more nucleotides, or (3) an insertion of one or more nucleotides. The term “altered” may be used to indicate a nucleotide or protein has been synthetically produced with a nucleotide or protein sequence that differs from wild-type. The term “mutant” may also refer to an alteration in the number of copies of a gene or in one or more of the elements that control its expression.

In certain embodiments, the one or more altered amino acids are randomized or randomly determined. In other embodiments, the one or more altered amino acids are derived from a second AAV strain that is not AAVx.

The “x” in “AAVx” may refer to any AAV strain (serotypes, variants, and capsid-engineered mutants). In certain embodiments, the first AAV strain, AAVx, is AAV2. In such embodiments, the plurality of antibodies may comprise anti-AAV2 capsid antibodies. In other embodiments, the first AAV strain, AAVx, is AAV9. The plurality of antibodies may comprise anti-AAV9 capsid antibodies.

In certain embodiments of the method of identifying a mutant AAV capsid protein, step (3), collecting the AAVx capsid mutants that bind to one or more antibodies, comprises immunoprecipitating the AAVx capsid mutants that bind to one or more antibodies. Examples are provided below.

Step (4) of the methods described herein, the identification of an AAVx capsid mutant that is indexed with a virus-specific barcode and that binds to one or more antibodies, may be performed as described herein or by using other Next-Generation DNA Sequencing (NGS) or other high-throughput sequencing methods.

The present disclosure also provides for the production of mutant AAV capsids that are identified using the methods described above; AAV vectors comprising such mutant AAV capsids; pharmaceutical compositions comprising such AAV vectors and a pharmaceutically acceptable adjuvant, excipient, carrier, or stabilizer; nucleic acid sequences that encode such mutant AAV capsids; and genetic constructs such as plasmids and viral genomes comprising such nucleic acid sequences. The AAV vectors described herein may be used to introduce genes into a mammalian cell, e.g., for gene therapy. Pharmaceutical compositions may be used for gene therapy, as vaccines, or for other therapeutic purposes.

The present disclosure also provides gene delivery vector products comprising a therapeutically effective amount of one or more of the AAV-derived capsids described herein and a pharmaceutically acceptable adjuvant, excipient, carrier, or stabilizer. The AAV-derived capsids may be derived from AAV2.

The gene delivery vector products and vaccines provided herein may comprise a pharmaceutically effective amount of at least one AAV-derived capsid or the novel AAV capsids as described herein and utilize suitable adjuvants, excipients, carriers and/or stabilizers known in the art to introduce one or more genes into a target cell or tissue or for inoculation to produce an immune response to a disease by stimulating the production of antibodies. The excipient, carrier and/or stabilizer useful in this invention are conventional and may include buffers, stabilizers, diluents, preservatives, and solubilizers. In general, the nature of the carrier or excipients will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g. powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

The adjuvant comprised in a vaccine may be selected from the group consisting of mineral oil-based adjuvants, preferably Freund's complete or incomplete adjuvant, Montanide incomplete Seppic adjuvants, preferably ISA, oil in water emulsion adjuvants, preferably Ribi adjuvant system, syntax adjuvant formulation containing muramyl dipeptide, and aluminum salt adjuvants.

In some embodiments the adjuvant is a mineral oil-based adjuvant, especially ISA206 (SEPPIC, Paris, France) or ISA51 (SEPPIC, Paris, France), or selected from the group consisting of CpG, Imidazoquinolines, MPL, MDP, MALP, flagellin, LPS, LTA, cholera toxin, a cholera toxin derivative, HSP60, HSP70, HSP90, saponins, QS21, ISCOMs, CFA, SAF, MF59, adamantanes, aluminum hydroxide, aluminum phosphate and a cytokine. In some embodiments, the composition, vaccine and/or gene delivery vector according to the invention comprises a combination of more than one, preferably two, adjuvants.

The term “therapeutically effective amount” or “pharmaceutically effective amount” refers to an amount that is sufficient to effect treatment, as defined below, when administered to a subject (e.g., a mammal, such as a human) in need of such treatment. The therapeutically or pharmaceutically effective amount will vary depending upon the subject and disease condition being treated, the weight and age of the subject, the severity of the disease condition, the manner of administration and the like, which can readily be determined by one of ordinary skill in the art. For example, a “therapeutically effective amount” or a “pharmaceutically effective amount” of AAV2-derived capsid as described herein is an amount sufficient to generate an immune response in a subject (e.g., a human). In some embodiments the immune response is sufficient to raise AAV capsid neutralizing antibodies against the relevant capsid(s) in the subject.

The lengths of scanning peptides may be of any length, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. In certain embodiments, the scanning peptides are between six to twelve amino acids in length. In certain embodiments, each AAVx capsid mutant comprises at least five, at least six, at least twelve, or between six and twelve altered amino acids.

The disclosure also provides AAVx-derived capsids comprising one or more mutations in an amino acid sequence of an epitope selected from at least one of Epitope 1, Epitope 2, Epitope 3, Epitope 4, Epitope 5, Epitope 6, Epitope 7, Epitope 8, Epitope 9, or Epitope 10. The mutated epitope amino acid sequence may be randomized. Alternatively, the mutated epitope amino acid sequence may be derived from an AAV strain other than AAVx. In certain embodiments, AAVx is AAV2.

In certain embodiments, the AAVx-derived capsids comprise one or more mutations in an amino acid sequence in an epitope selected from at least one of: Epitope 1: 439-DQYLYYLSRTNTPSGTTTQSRLQFSQAGASD-469 (SEQ ID NO:5); Epitope 2: 650-NTPVPANPSTTFSAAKFASFITQ-672 (SEQ ID NO:6); Epitope 3: 700-YTSNYNKSVNVDFTVDTNGVYSEPRPIGT-728 (SEQ ID NO:7); Epitope 4: 243-STRTWALPTYNNHLYKQISSQSGASNDNH-271 (SEQ ID NO:9); Epitope 5: 320-VKEVTQNDGTTTIANNLT-337 (SEQ ID NO:10); Epitope 6: 498-SEYSWTGATKYHLNGRDSL-516 (SEQ ID NO:11), Epitope 7: 523-MASHKDDEEKF-533 (SEQ ID NO:12); Epitope 8: 534-FPQSGVLIFGKQGSEKTNVDIEKVMIT-560 (SEQ ID NO:13); Epitope 9: 570-PVATEQYGSVSTNLQRGNRQAATADVN-596 (SEQ ID NO:8); or Epitope 10: 409-FTFSYTFEDVPFHS-422 (SEQ ID NO:52). AAVx may be AAV2. An AAVx-derived capsid as provided herein may be configured to escape antibody binding or neutralization.

The present disclosure also provides AAV vectors comprising such AAVx-derived capsids, and pharmaceutical compositions comprising a therapeutically effective amount of such AAV vectors and a pharmaceutically acceptable adjuvant, excipient, carrier, or stabilizer. The vectors and pharmaceutical compositions may be used for gene therapy or as a vaccine.

The present disclosure also provides an AAV capsid of an AAV strain comprising a mutant Epitope 1 amino acid sequence, wherein the mutant Epitope 1 amino acid sequence comprises GGTAATE (SEQ ID NO:14), TQEARPG (SEQ ID NO:20), TPTPQFS (SEQ ID NO:22), TLEPLIT (SEQ ID NO:24), PFETDLM (SEQ ID NO:26), LQEAHLT (SEQ ID NO:28), EEGGRPK (SEQ ID NO:29), EGDGGCL (SEQ ID NO:31), DGGAGSW (SEQ ID NO:32), AEGGGGG (SEQ ID NO:34), AGGGEMG (SEQ ID NO:36), GEAAAPA (SEQ ID NO:37), SVEGGAW (SEQ ID NO:38), or SLASTLE (SEQ ID NO:40). In certain embodiments, the AAV strain is AAV2. In certain other embodiments, the AAV strain is selected from the group consisting of AAV1, AAV3, AAV4, AAV5, AAV6, AAV7, AAV8, AAV9, AAV10, AAV11, AAV12, and AAV13. The AAV strain may also be another naturally occurring AAV variant or a capsid engineered mutant.

The present disclosure also provides an AAV capsid of an AAV strain comprising a mutant Epitope 2 amino acid sequence, wherein the mutant Epitope 2 amino acid sequence comprises PARQL (SEQ ID NO:15), PRPVQ (SEQ ID NO:19), PSALM (SEQ ID NO:21), ADSLL (SEQ ID NO:23), PASVM (SEQ ID NO:25), PRPLM (SEQ ID NO:27), AQPVM (SEQ ID NO:30), SEKQL (SEQ ID NO:33), APAMC (SEQ ID NO:35), DRRLL (SEQ ID NO:39), or TLPMK (SEQ ID NO:41). In certain embodiments, the AAV strain is AAV2. In certain other embodiments, the AAV strain is selected from the group consisting of AAV1, AAV3, AAV4, AAV5, AAV6, AAV7, AAV8, AAV9, AAV10, AAV11, AAV12, and AAV13. The AAV strain may also be another naturally occurring AAV variant or a capsid engineered mutant.

The present disclosure also provides an AAV capsid of an AAV strain comprising a mutant Epitope 3 amino acid sequence, wherein the mutant Epitope 3 amino acid sequence comprises SVDGN (SEQ ID NO:16). In certain embodiments, the AAV strain is AAV2. In certain other embodiments, the AAV strain is selected from the group consisting of AAV1, AAV3, AAV4, AAV5, AAV6, AAV7, AAV8, AAV9, AAV10, AAV11, AAV12, and AAV13. The AAV strain may also be another naturally occurring AAV variant or a capsid engineered mutant.

In certain embodiments, an AAV capsid as described above may be configured to escape antibody binding or neutralization.

The present disclosure also provides AAV vectors comprising any of the AAV capsids described above; pharmaceutical compositions comprising a therapeutically effective amount of one or more of these AAV vectors and a pharmaceutically acceptable adjuvant, excipient, carrier, or stabilizer; nucleic acid sequences that encodes such AAV capsids; and genetic constructs such as plasmids and viral genomes comprising such nucleic acid sequences. The AAV vectors described herein may be used to introduce genes into a mammalian cell, e.g., for gene therapy. Pharmaceutical compositions may be used for gene therapy, as vaccines, or for other therapeutic purposes.

The IP-Seq (Immunoprecipitation followed by AAV Barcode-Seq) procedure has been optimized using Protein A/G magnetic beads. This procedure is described in PCT international application No. PCT/US2015/027536, filed Apr. 24, 2015. An epitope in the AAV2 capsid that is recognized by the mouse monoclonal antibody against intact AAV2 particles (A20) has been mapped by IP-Seq. Epitopes in the AAV2 capsid have been mapped that are recognized by the mouse polyclonal antibodies developed in mice immunized by intravenous injection of an AAV2 vector. Strategies for the creation of anti-AAV neutralizing antibody-escaping AAV capsid mutants have been developed based on the new IP-Seq data.

The PK-Seq (Pharmacokinetic profiling by AAV Barcode-Seq) is a procedure by which AAV capsid neutralizing antibody epitopes can be identified through AAV-Barcode-Seq-based pharmacokinetic profiling of each AAV-HP or AAV-DP mutants. This procedure is described in PCT international application No. PCT/US2015/027536, filed Apr. 24, 2015. In brief, a DNA/RNA-barcoded AAV library composed of a set of AAV-HP or AAV-DP capsid mutants and a reference control AAV (e.g., DNA/RNA-barcoded dsAAV-U6-VBCLib libraries packaged with HP or DP scanning mutants) is incubated with human or animal sera in test tubes at 37° C. for one hour. The mixture of the AAV library and each serum sample is then injected intravenously into mice, and blood samples are collected at 1 min, 10 min, 30 min, 1 h and 4 h time points following injection. AAV viral genome DNA is extracted from each sample and subjected to the AAV Barcode-Seq analysis (Adachi K et al., Nat Commun 5, 3075 (2014)) to determine the blood clearance rate of each AAV strain contained in the AAV library.

AAV-HP or DP mutants whose HP or DP amino acid sequences contain an anti-AAV capsid neutralizing antibody epitope exhibit accelerated blood clearance when they are pre-incubated with human or animal sera containing anti-AAV neutralizing antibodies.

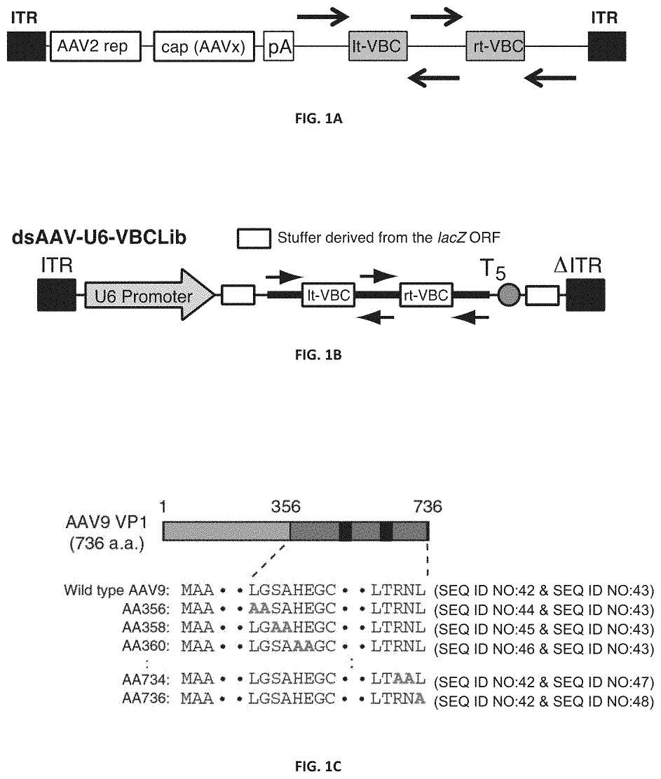

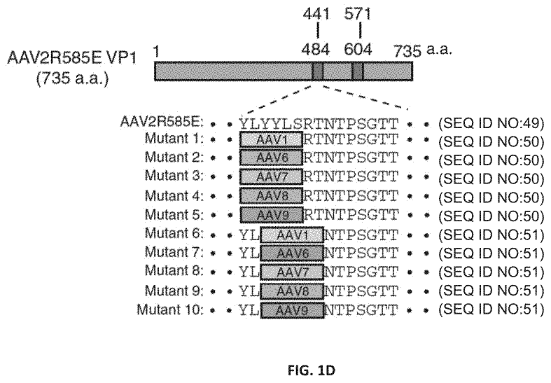

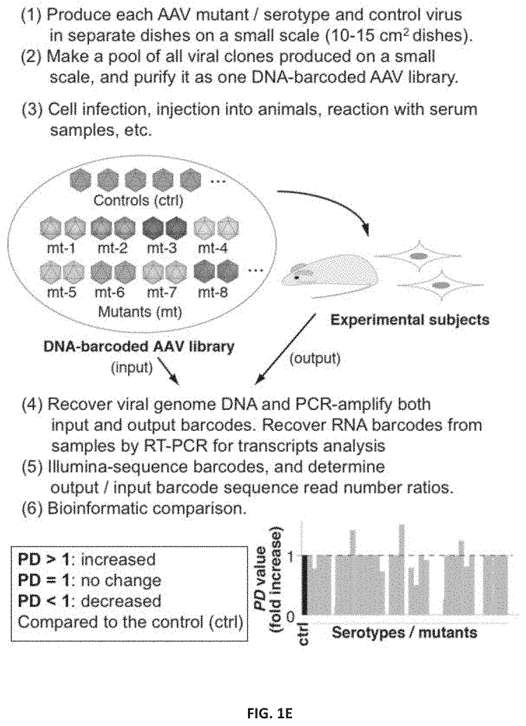

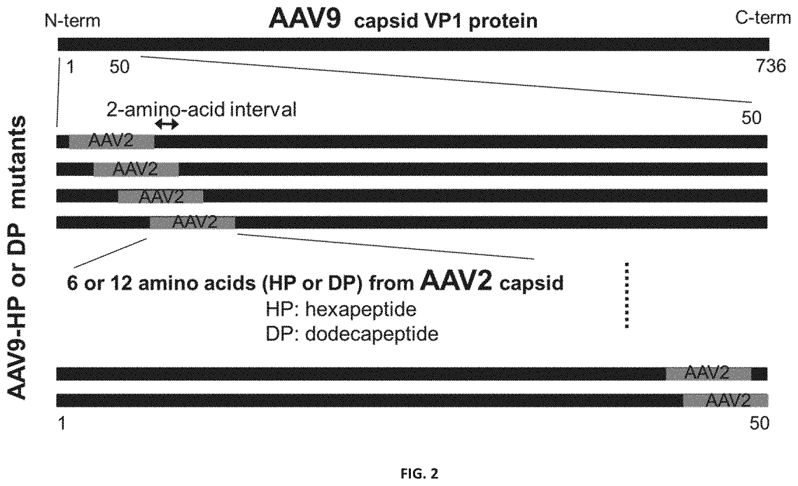

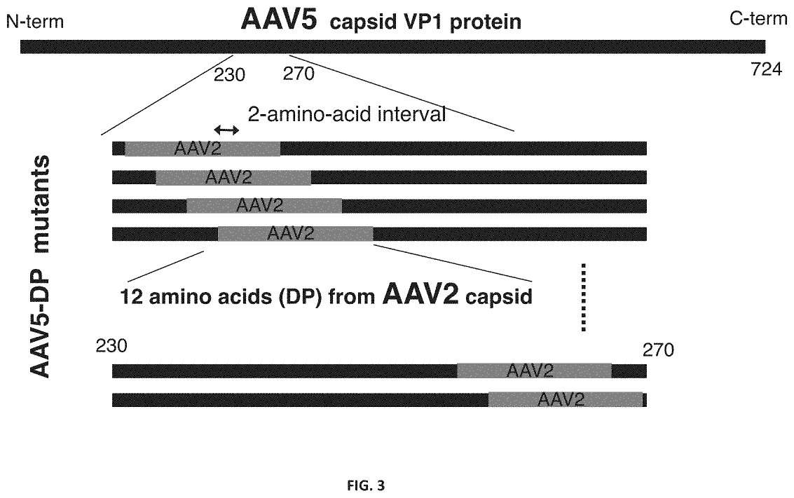

AAV Barcode-Seq, an NGS-based method that allows the characterization of phenotypes of hundreds of different AAV strains (i.e., naturally occurring serotypes and laboratory-engineered mutants) in a high-throughput manner with significantly reduced time and effort and using only a small number of subjects (e.g., tissue cultures and experimental animals), has recently been established (Adachi K et al., Nat Commun 5, 3075 (2014)). Using this approach, biological aspects including, but not limited to, blood clearance rate, transduction efficiency, tissue tropism, and reactivity to anti-AAV NtAbs can be assessed. FIGS. 1A-1E schematically depict the AAV Barcode-Seq approach. The principle of this approach is as follows. When a library stock comprising many different AAV strains is applied to certain types of samples (e.g., cells), the composition of the AAV population would in theory not change between the original input library and the library recovered from the samples if each of the AAV strains had exactly the same biological properties in a given context. However, if some strains show a different biological property (e.g., faster blood clearance or more efficient cellular internalization) compared to the others, there would be a change in the population composition between the input library (i.e., the library stock) and the output library (i.e., the library recovered from the samples). The basic method consists of a bioinformatic comparison between the input and output libraries using a similar principle as that employed in RNA-Seq (Wang Z et al., Nat Rev Genet 10, 57-63 (2009)). This method can allow the quantification of phenotypic differences between different AAV strains as a function of strain demographics. Such an analysis becomes possible by tagging each AAV strain with a unique short DNA barcode and applying ILLUMINA barcode sequencing to the resulting population (Smith A M et al., Genome Res 19, 1836-1842 (2009)).

A universal Barcode-Seq system expressing RNA barcodes, termed AAV DNA/RNA Barcode-Seq, has been devised (Adachi K et al., Mol Ther 22 (2014)). In this system, AAV libraries are produced in which each viral particle contains a DNA genome that is devoid of the rep and cap genes but is transcribed into an RNA barcode unique to its own capsid. This RNA barcode system, AAV DNA/RNA Barcode-Seq, has been employed for anti-AAV NtAb epitope mapping.

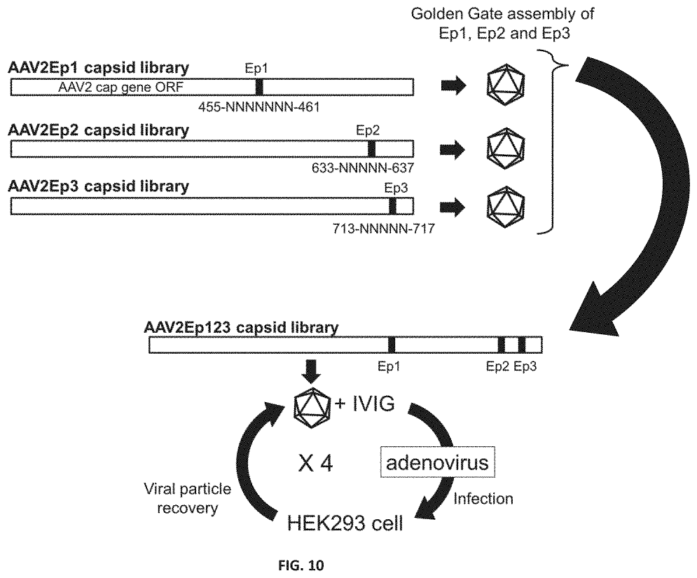

In this system, DNA/RNA-barcoded dsAAV-U6-VBCLib libraries packaged with HP scanning mutants can be produced. Such HP mutants can be AAV2R585E-HP scanning mutants for anti-AAVx NtAb epitope mapping (x=any strains other than AAV2 that do not cross-react with anti-AAV2 NtAb) and AAV9-HP scanning mutants for anti-AAV2 NtAb epitope mapping. The structure of AAV2R585E-HP mutants is shown in FIG. 10. AAV9-HP mutants are those in which AAV9 HPs are replaced with those derived from the AAV2 capsid.

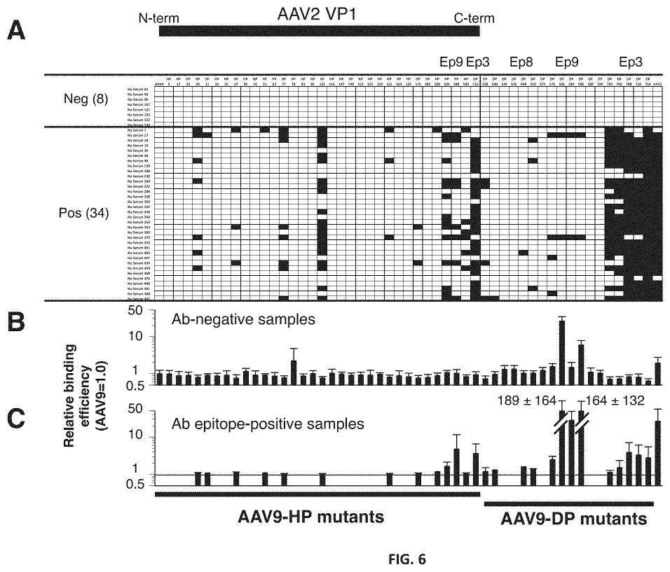

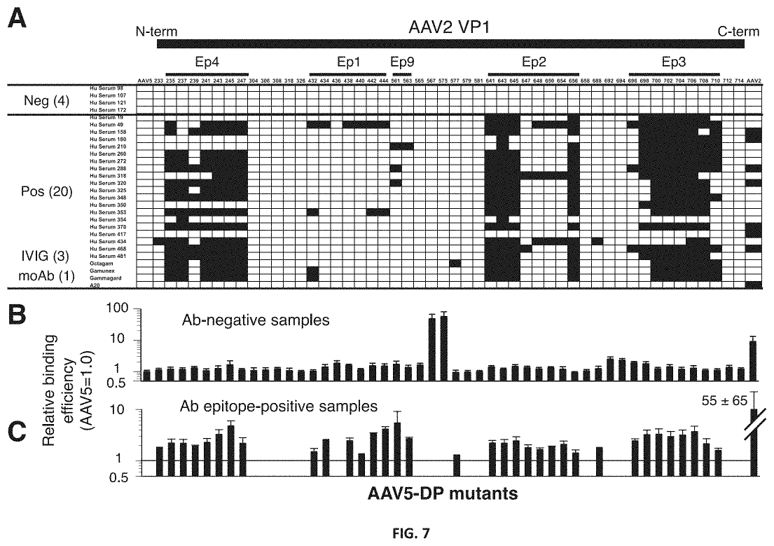

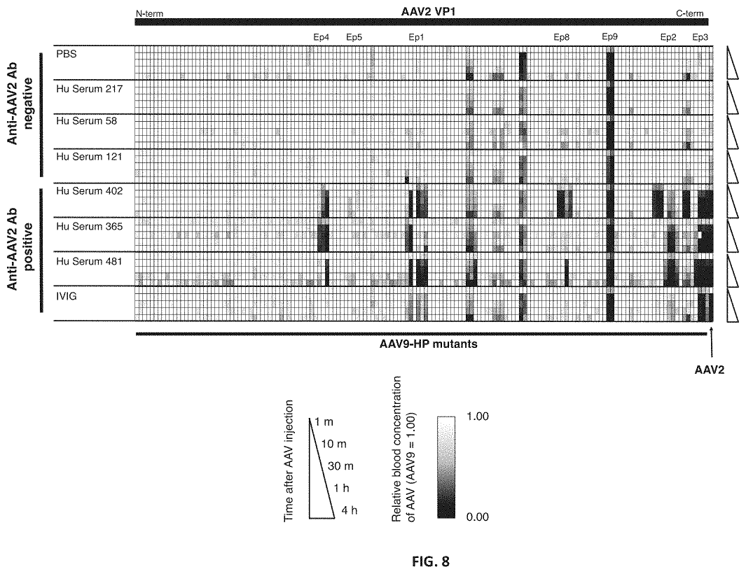

In place of hexapeptides (HPs), dodecapeptides (DPs) can also be utilized in the same manner for anti-AAVx NtAb epitope mapping. Many AAV9-DP mutants have been successfully produced as shown in FIG. 6 and FIG. 9.

In place of AAV9 as the launching platform serotype, AAV5 can also be utilized in the same manner for anti-AAVx NtAb epitope mapping. Many AAV5-DP mutants have been successfully produced as shown in FIG. 7. The less prevalence of anti-AAV5 antibodies in the human population than other common serotypes makes AAV5 an attractive platform for HP and DP scanning for antibody epitope mapping.

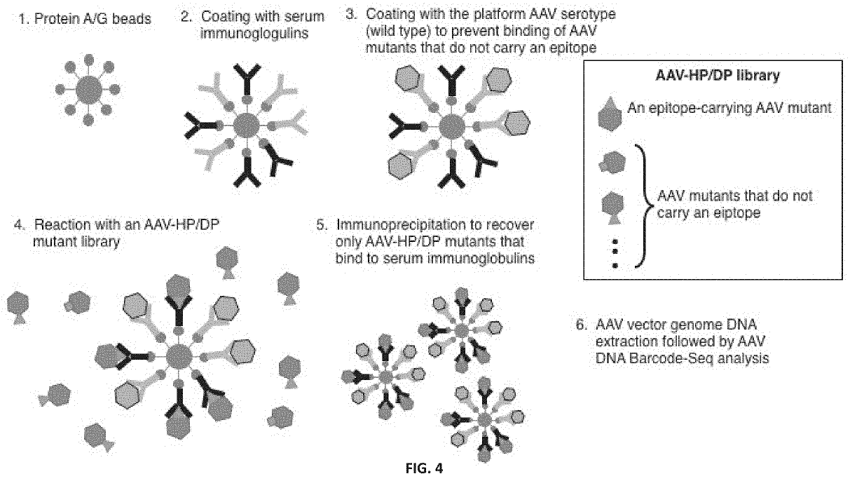

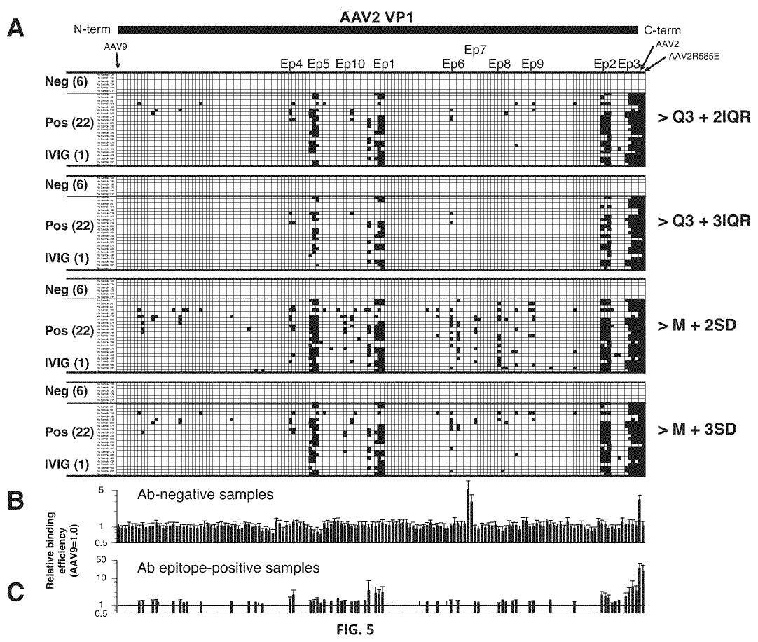

The IP-Seq based method does not require animals and is capable of mapping antibody epitopes of multiple samples at one time using multiplexed ILLUMINA sequencing. The procedure for IP-Seq based anti-AAV antibody epitope mapping can be as follows and is briefly explained in FIG. 4. First, 25 μl of serum samples (containing anti-AAV NtAbs) and 20 μl of PROTEIN A/G PLUS-AGAROSE (SANTA CRUZ sc-2003) can be incubated in a total volume of 100 μl in PBS in 1.5 ml tubes at 4° C. for 1 hour on a rotation device. After washing with PBS, a DNA/RNA-barcoded dsAAV-U6-VBCLib library and the agarose beads coated with immunoglobulins can be mixed in a total volume of 100 μl PBS, and may then be incubated at 4° C. overnight on a rotation device. On the next day, a standard IP procedure may be followed, the supernatants and immunoprecipitates can be collected and viral genome DNA can be extracted using a WAKO DNA Extraction Kit (such as the DNA Extractor® series of kits available from FUJIFILM Wako Pure Chemical Corp.) following Proteinase K treatment of the samples.

The subsequent procedure may be similar to that used for AAV Barcode-Seq as described in Adachi K et al., Nat Commun 5, 3075 (2014). Briefly, left and right viral clone-specific barcodes (It-VBC and rt-VBC in FIGS. 1A-1E) may be PCR-amplified using viral genome DNA recovered from the IP supernatants and precipitates. The PCR primers can be indexed with sample-specific DNA barcodes. All the PCR amplicons may then be mixed into a pool and the pool may be subjected to ILLUMINA sequencing. The ILLUMINA sequencing data may be bioinformatically analyzed to detect demographic changes of the AAV library in each sample. The principle of the method is that viral clones with higher avidity to sample immunoglobulins than others can be detected as clones that are decreased or depleted in the supernatants while enriched in the precipitates by ILLUMINA barcode sequencing. Such clones may likely carry epitopes for anti-AAV antibodies under investigation, and the epitopes targeted by the antibodies may likely be the heterologous peptides incorporated into the capsid of particular AAV clones showing a demographic change. 1×10⁷, 1×10⁸, and 1×10⁹ vg per 1.5 ml tube have been used. For the routine IP-Seq procedure, reliable and reproducible results can be obtained only from the DNA recovered from IP precipitates.

Here the IP-Seq procedure was utilized. In this procedure, a DNA-barcoded AAV9-hexapeptide (HP) scanning capsid mutant library was produced comprising a total of 153 AAV9-HP mutants in addition to the wild-type AAV9 (a negative control), as well as the wild-type AAV2 and the AAV2R585E heparin binding-deficient mutant (positive controls). Each AAV9-HP mutant contained a substitution of 6 consecutive amino acids derived from different regions of the wild-type AAV2 capsid so that various HP regions in the AAV2 capsid can be displayed on the heterologous AAV9 capsid in a nearly native quaternary structure. The HP scanning of the AAV2 capsid was performed at a two amino acid interval creating 153 overlapping HPs. These AAV9-HP mutants cover the majority of the AAV2 capsid amino acids that differ from those of the AAV2 capsid. AAV9-HP-584-00002 and AAV9-HP-586-00002 were poorly produced. These two mutants cover the heparin binding site of the AAV2 capsid, 585-RGNR-588, and therefore, the approach using AAV9-HP mutants is not able to determine whether the heparin binding site constitute an antibody epitope. This limitation could be overcome by applying the AAV9-DP mutant approach as described below.

The IP-Seq procedure can include of the following steps: (1) IP of the AAV9-HP library (AAV viral particles containing DNA-barcoded genomes) with monoclonal or polyclonal antibodies present in commercially available reagents or animal sera; (2) extraction of DNA-barcoded genomes from immunoprecipitates; and (3) ILLUMINA barcode sequencing of the recovered viral genomes followed by a bioinformatic analysis. Optimization experiments revealed that the combination of A/G protein-coated magnetic beads and blocking with 2% BSA was an optimal condition for lowering non-specific binding without restricting binding of the library clones. In the IP-Seq analysis, whether or not each mutant binds to test samples was determined based on PD values. When PD of a particular AAV-HP or AAV-DP mutant is identified as an extreme outlier among all the AAV strains contained in an AAV capsid mutant library, such an outlying mutant is considered as an AAV-HP or AAV-DP mutant that binds anti-AAV capsid antibody. Extreme outliers are defined as either of the following: (1) those that show PD values higher than the two times the interquartile range (IQR) from the third quartile (Q3) of all the PD values obtained from anti-AAV capsid antibody-negative serum samples obtained from the same species (i.e., >Q3+21QR); (2) >Q3+3IRQ, (3) >M (mean)+2SD (standard deviation) and (4) >M+3SD. The Q3+31QR is the most stringent cut-off and M+2SD is the least stringent cut-off among the four criteria for outliers. Although the four of the five most common epitopes can be readily identified by AAV9-HP-based IP-Seq no matter which criterion is used (FIG. 5), several epitopes that could be unambiguously identified by PK-Seq could not be identified by IP-Seq when the Q3+31QR was used. For example, Ep8 could not be identified as an epitope by IP-Seq when the Q3+31QR cut-off was used even though PK-Seq clearly revealed that Ep8 is a neutralizing antibody epitope. When the M+2SD cut-off was used, Ep8 could be readily identified. Thus, we selected the M+2SD cut-off for all the subsequent IP-Seq experiments.

Our choice of the M+2SD cut-off might increase false positive discovery rate (FDR) in the IP-Seq analysis compared to the Q3+31QR cut-off-based identification even though our choice can increase the power to identify epitopes. To help identify potential false positive and false negative signals, our IP-Seq analysis always accompanies two additional data (Panels B's and C's in all the IP-Seq data (FIGS. 5, 6, and 7) showing the ability for each mutant to bind to antibody-coated beads relative to that of the wild-type reference control (e.g., AAV9 or AAV5).

In these two additional dataset, the binding abilities were determined using anti-AAV antibody-negative human serum samples (Panel B's in FIGS. 5, 6, and 7) and samples positive for epitope signals (Panel C's in FIGS. 5, 6, and 7). Epitopes that show a relative binding efficiency of ˜1 (Panel B's in FIGS. 5, 6, and 7) in antibody-negative samples and that of much greater than 1 (Panel C's in FIGS. 5, 6, and 7) in antibody epitope signal-positive samples are most likely true epitopes. On the other hand, false positives could be contained among those that show a relative binding efficiency of close to 1. Thus some or many of the positive signals identified in the N-terminal region upstream of Ep4 may not represent true epitopes and were not reproducible in the two separate IP-Seq experiments that used different AAV9 mutant libraries (FIGS. 5 and 6).

Using the AAV9-HP mutant library and the IP-Seq procedure, amino acids were identified that are contained in the known epitope of the A20 mouse monoclonal antibody against intact AAV2 particles, which demonstrates proof-of-principle of the method. Subsequently, using the same approach, epitopes of polyclonal anti-AAV2 capsid antibodies were identified in the sera of AAV2-immunized mice. The identified epitopes include 261-SSQSGA-266 (SEQ ID NO:3) (the same as the epitope of A20) and 451-PSGTTT-456 (SEQ ID NO:4), which are shared with multiple serum samples.

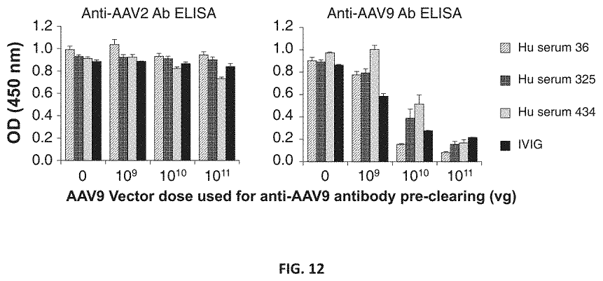

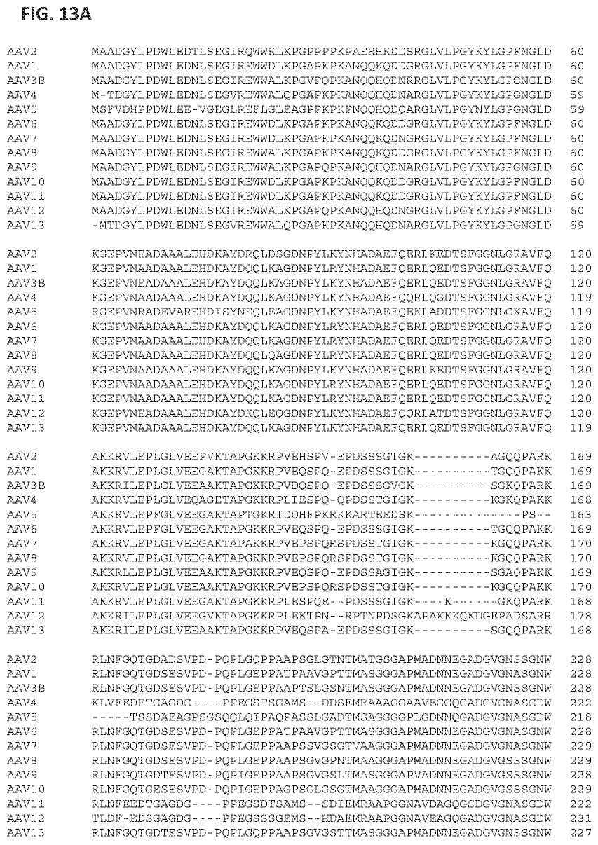

Initial ELISA screening of human sera has shown that many anti-AAV2 antibody-positive human serum samples are also positive for anti-AAV9 antibodies. This may make it difficult in some circumstances to apply the IP-Seq procedure directly to human samples because effective mapping of anti-AAV2 antibody epitopes is generally possible only when samples do not bind AAV9. To cope with this issue, an anti-AAV9 antibody neutralizing technique of incubating human sera with an excess amount of AAV9 particles before subjecting the sera to IP-Seq has been developed and confirmed by ELISA (FIG. 12). Accordingly, human sera may be screened to find suitable sera samples for IP-Seq and identification of polyclonal human antibody epitopes. The IP-Seq procedure can be an effective approach for mapping conformational anti-AAV capsid antibody epitopes and future development of anti-AAV neutralizing antibody-escaping mutants.

To determine the amount of AAV9 viral particles sufficient to neutralize anti-AAV9 capsid antibody activities present in 20 μl of human serum samples that had both anti-AAV2 and anti-AAV9 capsid antibodies, anti-AAV2 capsid antibody ELISA and anti-AAV9 capsid ELISA were performed using the human serum samples or IVIG that were pre-incubated with four different amounts of AAV9 vector particles, 0 vg, 1×10⁹ vg, 1×10¹⁹ vg or 1×10¹¹ vg at 37° C. for one hour. The pre-incubation of the samples with AAV9 did not significantly affect the anti-AAV2 antibody titers measured by the ELISA; however, anti-AAV9 antibody levels declined in a manner dependent on the dose of AAV9 vector particles used for pre-incubation. Pre-incubation with 1×10¹¹ vg of AAV9 was found to be sufficient to neutralize anti-AAV9 capsid antibodies present in human sera. With this result, we used 1×10¹¹ vg of AAV9 to neutralize anti-AAV9 capsid antibody activities before using for in vitro and in vivo studies.

The same pre-incubation approach was established and successfully used for IP-Seq using AAV5-DP libraries.

In addition to AAV9-HP mutant library, an AAV9-HP+DP library was also produced and used for epitope mapping. The DP scanning approach made it possible to produce AAV9 mutants that have the AAV2 capsid-derived 585-RGNR-588 heparin binding motif; i.e., AAV9-DP-582-00002 (H584L/S586R/A587G/Q588N/A589R/Q592A), AAV9-DP-584-00002 (H584L/S586R/A587G/Q588N/A589R/Q592A/G594A/W595D) and AAV9-DP-586-00002 (S586R/A587G/Q588N/A589R/Q592A/G594A/W595D/Q597N). The AAV9-HP+DP library used for this study contained 33 AAV9-HP mutants and 19 AAV9-DP mutants. AAV9-DP-578-00002 and AAV9-DP-580-00002 were poorly produced, and therefore, the data were not collected from these two mutants.

In addition to AAV9-HP and AAV9-DP mutant libraries, we constructed an AAV5-DP library that was used for FIG. 7. We constructed a total of 68 AAV5-DP mutant capsids (TABLE 4). Among those, 18 mutants did not produce or only yielded low titers, and therefore excluded in the AAV5-DP library.

Short amino acid sequences in the AAV2 capsid protein have been identified (using IP-Seq) that may constitute conformational epitopes for anti-AAV2 capsid polyclonal antibodies present in human sera. Viral neutralizing antibody NtAb epitope mapping can play a role in the development of new vaccines and drugs for the prevention and treatment of infectious diseases. Epitope mapping can also play a role in the development of novel gene delivery vectors that can escape from the host immune system. The identification of anti-AAV2 capsid polyclonal antibody epitopes that are shared with many individuals may help design novel vectors that evade the host immune response (an obstacle to effective in vivo gene therapy).

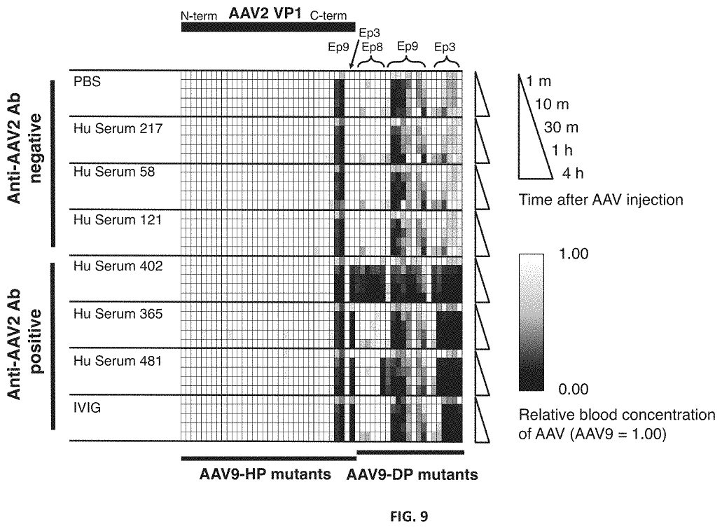

Previous studies using conventional approaches such as peptide scanning have yielded only a limited amount of information about human anti-AAV capsid epitopes. Using IP-Seq, however, five human anti-AAV2 capsid polyclonal antibody conformational epitopes (Ep1, Ep2, Ep3, Ep4 and Ep5) were identified that are shared by many individuals who have been infected with AAV2. In addition to these common epitopes, Ep6, Ep7, Ep8, Ep9, and Ep10 were also identified. Ep6, Ep7, Ep8, Ep9, and Ep10 are less common than Ep1, Ep2, Ep3, Ep4 and Ep5 but could be found in at least 5 out of 34 individuals positive for anti-AAV2 capsid antibody (see FIG. 5, FIG. 6, FIG. 7, FIG. 8, and FIG. 9) in at least either of the following assays, IP-Seq with M+2SD cut-off or PK-Seq. There is a gap within Ep1, Ep6, and Ep10 in the IP-Seq and PK-Seq epitope heatmaps (FIG. 5 and FIG. 8) however, we consider them as a single epitope because flanking epitope-containing scanning peptides overlap over the gap. Please note that we claim additional 5 amino acids adjacent to the N-terminal side of the identified epitopes and additional 5 amino acids adjacent to the C-terminal side of the identified epitopes as a part of epitopes. We have found that AAV5-DP-656-00002 carrying a DP, “ASFITQYSTGQV” (Ep3) has only one amino acid difference from the platform capsid at the N-terminal end of the DP (ASFITQYSTGQV vs. SSFITQYSTGQV). We have also found that AAV9-HP-524-00002 carrying a HP, “MASHKD” (Ep7) has only one amino acid difference from the platform capsid at the C-terminal end of the HP (MASHKD vs. MASHKE). Antigen-antibody interfaces require a contact of multiple amino acids; therefore, Ep3 and Ep7 should contain not only A and D but amino acids adjacent to the A and D residues in its tertiary or quaternary structure, respectively. Therefore, it is reasonable to include adjacent 5 amino acids at both sides of the scanning peptides as a potential part of identified epitopes.

To be explicit, the amino acid sequences shown as Ep1, 2, 3, 4, 5, 6, 7, 8, 9, and 10 are amino acid residues are those that are critical for forming antibody-binding epitopes but are not necessarily sufficient to constitute antibody-binding sites. To be more explicit, the amino acid sequences in Ep1, 2, 3, 4, 5, 6, 7, 8, 9, and 10, when altered by amino acid addition, deletion or substitution, would potentially lead to loss of the ability for the viral capsid to bind anti-AAV capsid antibodies.

As discussed above, the following five epitopes were identified: Ep1, Ep2, Ep3 Ep4 and Ep5. These are the common human anti-AAV capsid polyclonal antibody conformational epitopes shared with many individuals who have ever infected with AAV2. The amino acid sequences of these epitopes are as follows:

Ep1:
(SEQ ID NO: 5)
439-DQYLYYLSRTNTPSGTTTQSRLQFSQAGASD-469
Ep2:
(SEQ ID NO: 6)
650-NTPVPANPSTTFSAAKFASFITQ-672
Ep3:
(SEQ ID NO: 7)
700-YTSNYNKSVNVDFTVDTNGVYSEPRPIGT-728
Ep4:
(SEQ ID NO: 9)
243-STRTWALPTYNNHLYKQISSQSGASNDNH-271
Ep5:
(SEQ ID NO: 10)
320-VKEVTQNDGTTTIANNLT-337

Please note that the amino acid sequences indicated with bold letters with an underline are epitopes identified by either or both of IP-Seq and PK-Seq, and additional 5 amino acids added to each of the N-terminal and C-terminal ends of the epitopes are amino acids that may contain the epitope as explained above.

Other human anti-AAV capsid polyclonal antibody conformational epitopes that were found in at least five out of 34 human serum samples containing anti-AAV2 capsid antibodies include:

Ep6:
(SEQ ID NO: 11)
498-SEYSWTGATKYHLNGRDSL-516
Ep7:
(SEQ ID NO: 12)
523-MASHKDDEEKF-533
Ep8:
(SEQ ID NO: 13)
534-FPQSGVLIFGKQGSEKTNVDIEKVMIT-560
Ep9:
(SEQ ID NO: 8)
570-PVATEQYGSVSTNLQRGNRQAATADVN-596
Ep10:
(SEQ ID NO: 52)
409-FTFSYTFEDVPFHS-422

Although Ep9, the sequence of which has been determined with AAV9-HP-582-00002 and AAV9-HP-588-00002, was found less commonly in the study, the approach used in this study was inconclusive in determining the actual frequency of Ep9 being an epitope. This is because AAV9-HP-584-00002 and AAV9-HP-586-00002 mutants were poorly produced and therefore were not able to provide information about epitopes.

That being said, this issue has been partially overcome by using AAV9-DP mutants (FIG. 6).

These amino acid regions (Ep1, 2, 3, 4, 5, 6, 7, 8, 9 and 10) are epitopes that can be recognized by human anti-AAV2 capsid polyclonal antibodies. By using an approach in which amino acid sequences in these regions are first randomized and subsequently selected for those that no longer bind antibodies by means of directed evolution, it may be possible to create novel AAV2-derived capsids that can escape antibody neutralization.

To show proof-of-principle of the above-described directed evolution approach to create novel antibody-escaping AAV2-derived mutant capsids, a directed evolution experiment using an AAV2Ep123 capsid library was performed on human kidney embryonic (HEK) 293 cells (FIG. 10). The AAV2Ep123 capsid mutant library contained diverse mutants whose 7-mer, 5-mer and 5-mer peptide sequences in the Ep1, Ep2 or Ep3 epitope region of the AAV2 capsid were randomized.

The AAV2Ep123 capsid mutant library was constructed as follows. AAV2Ep1 capsid mutant library, AAV2Ep2 capsid mutant library and AAV2Ep3 capsid mutant library were independently produced in HEK293 cells. The Ep1, Ep2 and Ep3 coding regions of the viral genome DNA extracted from the produced viral particles were first PCR-amplified separately, and joined randomly by the Golden Gate assembly. The resulting recombinant DNA was used to produce the AAV2Ep123 capsid mutant library in HEK293 cells (FIG. 10).

The AAV2Ep123 capsid mutant library was first incubated with IVIG containing neutralizing antibodies against various AAV serotypes including AAV2. The IVIG-treated AAV2Ep123 capsid mutant library was then applied on HEK293 cells in the presence of adenovirus type 5. The amplified AAV mutant viral particles in HEK293 cells were recovered from and used for the next round selection on HEK293 cells. A total of four rounds of selection were performed to obtain AAV2Ep123 mutants resistant to neutralization by anti-AAV capsid antibodies.

This directed evolution experiment identified at least 16 AAV2Ep123 mutants with AAV2Ep123mt1 being most enriched (Table 4). This mutant was the only mutant that carried non-native amino acid sequence in the Ep3 epitope position. All the other mutants, AAV2Ep1mt2 to mt16, had the wild-type sequence in the Ep3 epitope region, indicating that the Ep3 region is not as tolerant to amino acid changes as the Ep1 or Ep2 region. The AAV2Ep123mt1 carries GGTAATE (SEQ ID NO:14) for Ep1, PARQL (SEQ ID NO:15) for Ep2 and SVDGN (SEQ ID NO:16) for Ep3.

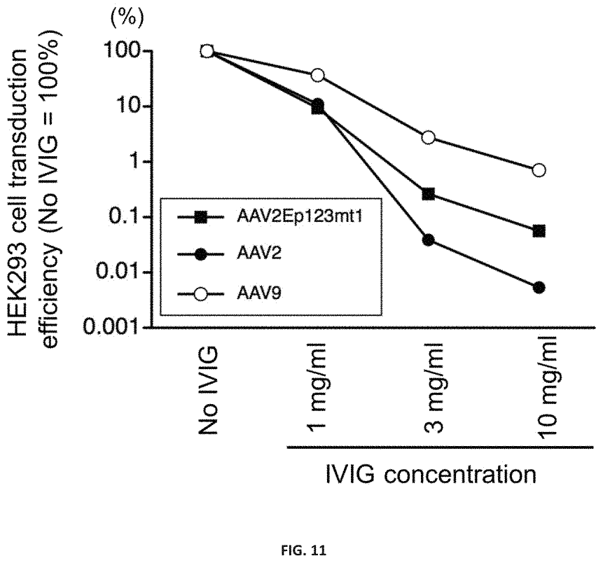

The ability for AAV2Ep123mt to escape from antibody-mediated neutralization was assessed by two independent sets of in vitro cell culture experiment. 1×10⁹ vector genomes (vg) of AAV vector particles (AAV2-CMV-luc or AAV2Ep123mt1-CMV-luc) were reacted with 10 μl of IVIG at varying concentrations (1, 3 and 10 mg/ml) at 37° C. for one hour, and the remaining viral infectivity was assessed by measuring luciferase activity using a luminometer. AAV2-CMV-luc and AAV2Ep123mt1-CMV-luc are AAV2 vectors expressing a firefly luciferase under the control of the human cytomegalovirus (CMV) immediately early enhancer-promoter. The result showed that AAV2Ep123mt is approximately 7 and 11-fold resistant to neutralization by IVIG at 3 and 10 mg/ml, respectively (FIG. 11).

In addition to the directed evolution approach described above, this information can be utilized for other types of AAV capsid engineering. The IP-Seq and PK-Seq approaches can be applied to other AAV serotypes or mutants for the identification of human anti-AAV capsid polyclonal antibody conformational epitopes.

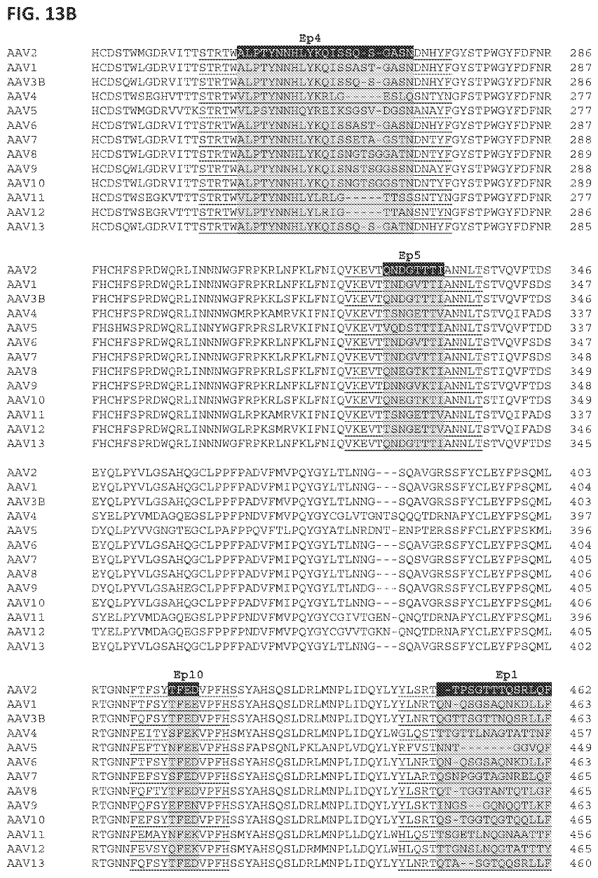

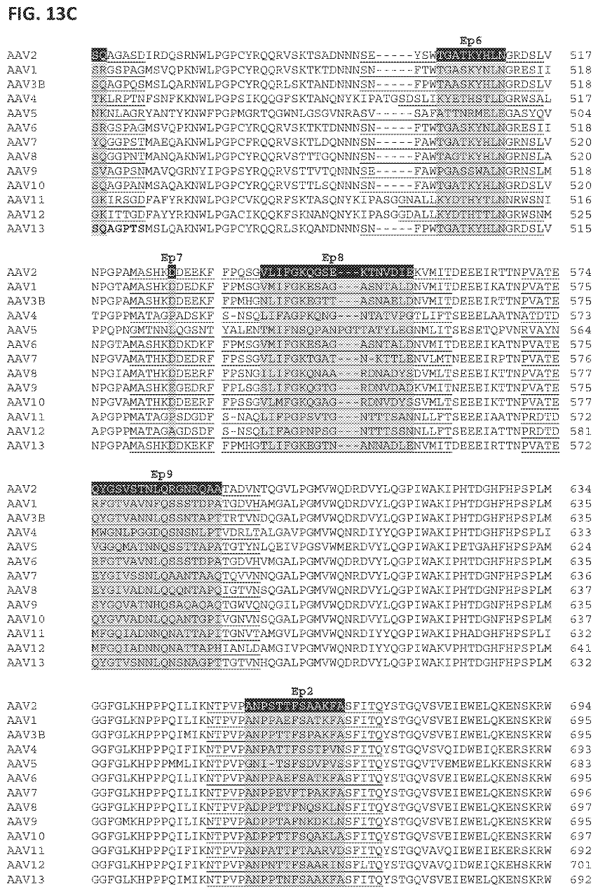

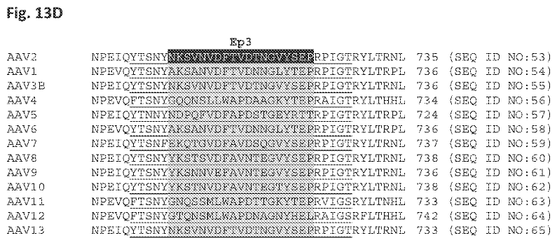

As noted above, the proof-of-principle of viral neutralizing antibody (NtAb) epitope mapping using barcoded hexapeptide (HP) or dodecapeptide (DP) scanning library in a high-throughput manner was established in the context of AAV.Sequencing alignment of the VP proteins of AAV1, 2, 3B, 4, 5, 6, 7, 8, 9, 10, 11, 12 and 13 using Clustal Omega reveals potential anti-AAV capsid antibody epitopes of AAV capsids derived from non-AAV2 serotypes (FIGS. 13A, 13B, 13C, 13D). This information can be exploited to develop novel AAV capsids derived from various serotypes and capsid mutants that are more resistant to NtAbs.

### EXAMPLES

The following examples are illustrative of disclosed methods. In light of this disclosure, those of skill in the art will recognize that variations of these examples and other examples of the disclosed method would be possible without undue experimentation.

### Example 1

553 human serum samples were collected from the Oregon Health & Science University (OHSU) blood lab and were screened for anti-AAV2 capsid antibodies by ELISA. Up to 34 human serum samples that showed high antibody titers by ELISA were subjected to IP-Seq and anti-AAV2 capsid polyclonal antibody conformational epitopes were determined.

### Example 2

A DNA/RNA-barcoded dsAAV-U6-VBCLib library packaged with the AAV9-HP scanning mutants was produced (see Table 1). This library, termed dsAAV9-HP-U6-VBCLib, contained 153 AAV9-HP mutants (2 clones per mutant), AAV2 (2 clones) and the two reference controls, AAV2R585E and AAV9 (15 clones each). PIERCE™ PROTEIN A/G MAGNETIC BEADS were incubated with human serum samples to coat the beads with anti-AAV2 capsid antibodies. Then the anti-AAV2 antibody-coated beads were incubated with the dsAAV9-HP-U6-VBCLib library. By a standard immunoprecipitation procedure, AAV clones bound to the beads were precipitated. The viral DNA from the precipitated viral particles was extracted and subjected to the AAV Barcode-Seq analysis (Adachi, et al. Nature Communications 5:3075, 2014.). All the values were normalized with the values obtained from the AAV9 reference controls.

### Example 3

Another DNA/RNA-barcoded dsAAV-U6-VBCLib library packaged with the AAV9-HP and AAV9-DP scanning mutants was produced (see Table 2). This library, termed dsAAV9-HP+DP-U6-VBCLib, contained 33 AAV9-HP mutants (2 clones per mutant), 19 AAV9-DP mutants (2 clones per mutant), AAV2 (5 clones) and one reference control, AAV9 (15 clones). The IP-Seq analysis using this library was performed in the same manner described above.

### Example 4

The above-described dsAAV9-HP-U6-VBCLib and dsAAV9-HP+DP-U6-VBCLib library-based IP-Seq approach can identify anti-AAV2 capsid antibody epitopes. However, it is not possible to determine whether or not the antibodies that bind the epitopes identified by IP-Seq have the ability to neutralize AAV infection. To address this limitation, the in vivo PK-Seq approach using dsAAV9-HP-U6-VBCLib or dsAAV9-HP+DP-U6-VBCLib library was developed. The concept of this in vivo approach has been described in PCT international application No. PCT/US2015/027536, filed Apr. 24, 2015. Fifty μl of anti-AAV capsid antibody-containing samples (3 anti-AAV2 capsid antibody-positive human serum samples and 10 mg/ml IVIG) or antibody-negative control samples (3 anti-AAV2 antibody-negative human serum samples and PBS) were incubated with 1×10¹¹ vg of AAV9-CMV-lacZ in 20 μl at 37° C. for 1 hour to neutralize anti-AAV9 capsid antibody activities followed by additional 1-hour incubation with 1×10⁹ vg of dsAAV9-HP-U6-VBCLib or dsAAV9-HP+DP-U6-VBCLib in 20 After the completion of ex vivo incubation, the sample volume was brought up to 300 μl using PBS/5% sorbitol. Eight-week-old C57BL/6 male mice were injected via the tail vein with the above-described 300 μl mixture as a bolus. Blood samples were collected 1 min, 10 min, 30 min, 1 hour and 4 hour post-injection, and subjected to the AAV Barcode-Seq analysis. AAV9-HP and AAV9-DP mutants that carry an anti-AAV2 capsid antibody epitope were cleared from the bloodstream significantly faster when pre-incubated with the samples containing anti-AAV2 capsid antibodies than when pre-incubated with the samples containing no anti-AAV2 capsid antibodies (FIG. 8 and FIG. 9). The data were collected from 2 mice per sample. The PK-Seq analysis revealed that Ep5 identified in three human samples (ID365, ID402 and ID481) are not neutralizing epitopes while Ep1, Ep2, Ep3, Ep4, Ep8 and Ep9 are neutralizing epitopes. In addition, PK-Seq could identify epitopes that IP-Seq failed to identify (e.g., Ep4 and Ep8 in sample ID365 and ID481). Thus, PK-Seq complements IP-Seq, provides more sensitive detection of antibody epitopes, and differentiates neutralizing and non-neutralizing antibody epitopes. However, 6 AAV9-HP and 3 AAV9-DP mutants were cleared from the blood circulation very rapidly following intravenous infusion even in the absence of anti-AAV2 antibodies in mice. This made it difficult to assess epitopes for these 9 mutants.

### Example 5

The anti-AAV2 capsid antibody-positive human serum samples that also had anti-AAV9 antibodies were precleared with pre-incubation with AAV9 viral particles to remove anti-AAV9 polyclonal antibodies from the samples (FIG. 12). In the IP-Seq analysis shown in FIG. 5, four different criteria were used to define as positive (i.e., AAV9-HP or AAV9-DP mutants that bind to the beads). In the top and the second top panels in FIG. 5, the values showing more than two and three times the interquartile range (IQR) beyond the upper quartile (>Q3+21QR and >Q3+31QR) obtained from the antibody-negative samples were considered as positive (i.e., AAV9-HP or AAV9-DP mutants that bind to the beads), and shown as black boxes, respectively. In the bottom and the second bottom panels in FIG. 5, the values showing more than two and three times the standard deviation beyond the mean value (>M+2SD and >M+3SD) obtained from the antibody-negative samples were considered as positive and shown as black boxes, respectively. Five common epitopes shared with many samples (Ep1, Ep2, Ep3, Ep4 and Ep5) were found and five epitopes that are less common but shared with at least 5 samples (Ep6, Ep7, Ep8, Ep9, and Ep10) were also found. The top six rows in FIG. 5 indicates human sera with no anti-AAV2 antibody assessed by anti-AAV2 capsid antibody ELISA. It has been found that two AAV9-HP mutants, 514-00002 and 516-00002, and the wild-type AAV2 can bind to the IP beads nonspecifically in the absence of anti-AAV2 capsid antibodies, making a high background signals from anti-AAV2 capsid antibody-negative human serum samples (FIG. 5B). It should be taken into account that high background could compromise the power to detect true positives. Binding efficiency of each AAV strain (AAV9-HP mutants and AAV2 positive control) relative to that of AAV9 provide information useful to interpret the IP-Seq data although the data does not provide definitive conclusions (FIG. 5C). That is, high values (i.e., significantly higher than 1.0) strongly indicate true epitopes although positives with low values (i.e., close to 1.0) do not necessarily exclude the possibility that they are true epitopes.

### Example 6

The common epitopes that could be identified by IP-Seq using AAV9-HP mutants were also identified by IP-Seq using AAV9-DP mutants. We found that the IP-Seq using AAV9-DP has several advantages over the IP-Seq using AAV9-HP mutants. First, 3 out of the 4 AAV9 capsid mutants that contain the heparin binding site of the AAV2 capsid, 585-RGNR-588, could be produced at levels sufficient for the downstream IP-Seq procedure. That is, among AAV9-DP580-00002, AAV9-DP582-00002, AAV9-DP584-00002 and AAV9-DP586-00002, which contain 585-RGNR-588, only AAV9-DP580-00002 was poorly produced. Second, the IP-Seq using AAV9-DP mutants has a better ability to identify true epitopes. For example, the higher sensitivity was evidenced in identifying Ep8 as an epitope. PK-Seq identified Ep8 as an unambiguous neutralizing antibody epitope for the human samples ID402 and ID481 (FIG. 8 and FIG. 9). The IP-Seq using AAV9-DP could also reveal that Ep8 is an epitope for these samples (FIG. 6) while the IP-Seq using AAV9-HP mutants failed to identify Ep8 as an epitope (FIG. 5).

### Example 7

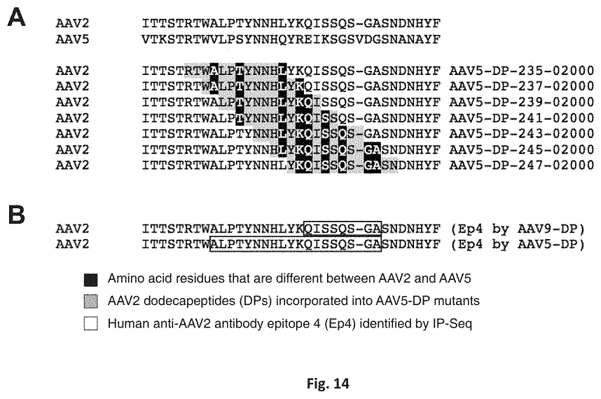

The common epitopes that could be identified by IP-Seq using AAV9-HP or AAV9-DP mutants were also identified by IP-Seq using AAV5-DP mutants (FIG. 7). We found that the AAV5-DP mutant approach complements the AAV9-HP and AAV9-DP mutant approaches in that the AAV5-DP approach could identify epitopes that AAV9-HP and AAV9-DP approach failed to identify. For example, AAV5-DP-235-00002, AAV5-DP-237-00002, AAV5-DP-239-00002, AAV5-DP-241-00002, AAV5-DP-243-00002, AAV5-DP-245-00002, and AAV5-DP-247-00002 (FIG. 14A) were precipitated as outliers by IP-Seq with anti-AAV2 antibody-positive human serum samples, which led to identifying ALPTYNNHLYKQISSQSGA are amino acids that comprise Ep4. However, the ALPTYNNHLYK sequence, the left half of Ep4, could not be identified as a part of Ep4 by the IP-Seq using AAV9-HP mutants (FIG. 14B).

### Example 8

As exemplified by the procedure that generated a set of AAV2Ep123mt mutants aimed at identifying anti-AAV2 neutralizing antibody-escaping AAV2 mutants, the epitope information can be exploited to develop novel mutants derived from any AAV strains (common serotypes, various natural variants and capsid-engineered mutants) that can evade pre-existing immunity. An example of the procedure is as follows: (1) Randomize or rationally modify amino acids in each common neutralizing epitope; (2) Perform directed evolution or screening of AAV capsid mutants containing an amino acid sequence-altered single epitope or a combination of two or more amino acid sequence-altered epitopes using an appropriate method in the presence or absence of appropriate anti-AAV neutralizing antibodies; (3) Perform further directed evolution or screening of AAV capsid mutants containing a combination of sequence-altered epitopes selected by the procedure (2) using an appropriate method in the presence or absence of appropriate anti-AAV neutralizing antibodies; and (4) Assess the ability of each selected AAV capsid mutant to escape from anti-AAV antibody-mediated neutralization and transduce target cells in cultured cells or target organs in animals using an appropriate method.

TABLE 1
Hexapeptide Scanning AAV9-derived Mutants

| Name of mutant¹ | Amino acid substitutions |
|---|---|
| AAV9-HP-009-00002 | N14T |
| AAV9-HP-017-00002 | E21Q |
| AAV9-HP-019-00002 | E21Q/A24K |
| AAV9-HP-023-00002 | A24K |
| AAV9-HP-025-00002 | A29P |
| AAV9-HP-027-00002 | A29P/Q31P |
| AAV9-HP-029-00002 | A29P/Q31P/A34P |
| AAV9-HP-031-00002 | Q31P/A34P/N35A/Q36E |
| AAV9-HP-033-00002 | A34P/N35A/Q36E/Q37R |
| AAV9-HP-035-00002 | N35A/Q36E/Q37R/Q39K |
| AAV9-HP-037-00002 | Q37R/Q39K/N41D/A42S |
| AAV9-HP-039-00002 | Q39K/N41D/A42S |
| AAV9-HP-041-00002 | N41D/A42S |
| AAV9-HP-051-00002 | G56F |
| AAV9-HP-063-00002 | A67E |
| AAV9-HP-077-00002 | Q81R |
| AAV9-HP-079-00002 | Q81R/K84D |
| AAV9-HP-081-00002 | Q81R/K84D/A85S |
| AAV9-HP-083-00002 | K84D/A85S |
| AAV9-HP-085-00002 | A85S |
| AAV9-HP-121-00002 | L125V |
| AAV9-HP-131-00002 | A135P/A136V |
| AAV9-HP-143-00002 | Q148H |
| AAV9-HP-147-00002 | Q148H/Q151V |
| AAV9-HP-149-00002 | Q151V |
| AAV9-HP-153-00002 | A157S |
| AAV9-HP-155-00002 | A157S/I159T |
| AAV9-HP-157-00002 | A157S/I159T/S162A |
| AAV9-HP-159-00002 | I159T/S162A/A164Q |
| AAV9-HP-161-00002 | S162A/A164Q |
| AAV9-HP-163-00002 | A164Q/K168R |
| AAV9-HP-165-00002 | K168R |
| AAV9-HP-175-00002 | T179A/E180D |
| AAV9-HP-183-00002 | I188L |
| AAV9-HP-185-00002 | I188L/E190Q |
| AAV9-HP-189-00002 | E190Q |
| AAV9-HP-193-00002 | V198L |
| AAV9-HP-195-00002 | V198L/S200T |
| AAV9-HP-197-00002 | V198L/S200T/L201N |
| AAV9-HP-199-00002 | S200T/L201N |
| AAV9-HP-201-00002 | L201N/S205T |
| AAV9-HP-203-00002 | S205T/G207S |
| AAV9-HP-207-00002 | G207S/V211M |
| AAV9-HP-209-00002 | V211M |
| AAV9-HP-219-00002 | S223N |
| AAV9-HP-229-00002 | Q233T |
| AAV9-HP-231-00002 | Q233T/L235M |
| AAV9-HP-235-00002 | L235M |
| AAV9-HP-257-00002 | N262S/S263 |
| AAV9-HP-259-00002 | N262S/S263/T264Q |
| AAV9-HP-261-00002 | N262S/S263/T264Q/G267/G268A |
| AAV9-HP-264-00002 | T264Q/G267/S268A |
| AAV9-HP-267-00002 | S268A |
| AAV9-HP-269-00002 | A273H |
| AAV9-HP-323-00002 | D327Q |
| AAV9-HP-325-00002 | D327Q/N329D |
| AAV9-HP-327-00002 | D327Q/N329D/V331T/K332T |
| AAV9-HP-329-00002 | N329D/V331T/K332T |
| AAV9-HP-331-00002 | V331T/K332T |
| AAV9-HP-345-00002 | D349E |
| AAV9-HP-357-00002 | E361Q |
| AAV9-HP-369-00002 | I374V |
| AAV9-HP-379-00002 | D384N |
| AAV9-HP-407-00002 | Q412T |
| AAV9-HP-411-00002 | Q412T/E416T |
| AAV9-HP-413-00002 | E416T |
| AAV9-HP-415-00002 | E416T/N419D |
| AAV9-HP-417-00002 | N419D |
| AAV9-HP-445-00002 | K449R |
| AAV9-HP-447-00002 | K449R/I451N/N452T |
| AAV9-HP-449-00002 | K449R/I451N/N452T/G453PS |
| AAV9-HP-451-00002 | I451N/N452T/G453PS/S454G/G455T |
| AAV9-HP-453-00002 | G453PS/S454G/G455T/Q456T/N457T |
| AAV9-HP-454-00002 | S454G/G455T/Q456T/N457T/Q459S |
| AAV9-HP-456-00002 | Q456T/N457T/Q459S/T460R |
| AAV9-HP-458-00002 | Q459S/T460R/K462Q |
| AAV9-HP-460-00002 | T460R/K462Q/V465Q |
| AAV9-HP-462-00002 | K462Q/V465Q |
| AAV9-HP-464-00002 | V465Q/P468A |
| AAV9-HP-466-00002 | P468A/N470D/M471I |
| AAV9-HP-468-00002 | P468A/N470D/M471I/A472R/V473D |
| AAV9-HP-470-00002 | N470D/M471I/A472R/V473D/G475S |
| AAV9-HP-472-00002 | A472R/V473D/G475S |
| AAV9-HP-474-00002 | G475S/Y478W/I479L |
| AAV9-HP-476-00002 | Y478W/I479L |
| AAV9-HP-478-00002 | Y478W/I479L/S483C |
| AAV9-HP-480-00002 | S483C |
| AAV9-HP-486-00002 | T491K |
| AAV9-HP-488-00002 | T491K/V493S |
| AAV9-HP-490-00002 | T491K/V493S/T494A/Q495D |
| AAV9-HP-492-00002 | V493S/T494A/Q495D |
| AAV9-HP-494-00002 | T494A/Q495D |
| AAV9-HP-496-00002 | F501Y |
| AAV9-HP-498-00002 | F501Y/A502S |
| AAV9-HP-500-00002 | F501Y/A502S/P504T |
| AAV9-HP-502-00002 | A502S/P504T/S507T |
| AAV9-HP-504-00002 | P504T/S507T/S508K/W509Y |
| AAV9-HP-506-00002 | S507T/S508K/W509Y/A510H |
| AAV9-HP-508-00002 | S508K/W509Y/A510H |
| AAV9-HP-510-00002 | A510H/N515D |
| AAV9-HP-512-00002 | N515D |
| AAV9-HP-514-00002 | N515D/M518V |
| AAV9-HP-516-00002 | M518V |
| AAV9-HP-524-00002 | E529D |
| AAV9-HP-526-00002 | E529D/G530D |
| AAV9-HP-528-00002 | E529D/G530D/D532E/R533K |
| AAV9-HP-530-00002 | G530D/D532E/R533K |
| AAV9-HP-532-00002 | D532E/R533K/L537Q |
| AAV9-HP-534-00002 | L537Q |
| AAV9-HP-536-00002 | L537Q/S540V |
| AAV9-HP-538-00002 | S540V |
| AAV9-HP-544-00002 | T548S/G549E |
| AAV9-HP-546-00002 | T548S/G549E/R550K/D551T |
| AAV9-HP-550-00002 | R550K/D551T/A555I |
| AAV9-HP-552-00002 | A555I/D556E |
| AAV9-HP-556-00002 | D556E |
| AAV9-HP-558-00002 | N562D |
| AAV9-HP-562-00002 | N562D/K567R |
| AAV9-HP-564-00002 | K567R |
| AAV9-HP-572-00002 | S576Q |
| AAV9-HP-574-00002 | S576Q/Q579S |
| AAV9-HP-576-00002 | S576Q/Q579S/A581S |
| AAV9-HP-578-00002 | Q579S/A581S |
| AAV9-HP-580-00002 | A581S/H584L |
| AAV9-HP-582-00002 | H584L/S586R/A587G |
| AAV9-HP-584-00002 | H584L/S586R/A587G/Q588N/A589R |
| AAV9-HP-586-00002 | S586R/A587G/Q588N/A589R |
| AAV9-HP-588-00002 | Q588N/A589R/Q592A |
| AAV9-HP-590-00002 | Q592A/G594A/W595D |
| AAV9-HP-592-00002 | Q592A/G594A/W595D/Q597N |
| AAV9-HP-594-00002 | G594A/W595D/Q597N/N598T |
| AAV9-HP-596-00002 | Q597N/N598T/I601V |
| AAV9-HP-598-00002 | N598T/I601V |
| AAV9-HP-600-00002 | I601V |
| AAV9-HP-624-00002 | N628H |
| AAV9-HP-636-00002 | M640L |
| AAV9-HP-652-00002 | D657N |
| AAV9-HP-654-00002 | D657N/P659S |
| AAV9-HP-656-00002 | D657N/P659S/A661T |
| AAV9-HP-658-00002 | P659S/A661T/N663S |
| AAV9-HP-660-00002 | A661T/N663S/K664A/D665A |
| AAV9-HP-662-00002 | N663S/K664A/D665A/L667F |
| AAV9-HP-664-00002 | K664A/D665A/L667F/N668A |
| AAV9-HP-666-00002 | L667F/N668A |
| AAV9-HP-668-00002 | N668A |
| AAV9-HP-702-00002 | Y706N |
| AAV9-HP-704-00002 | Y706N/N709V |
| AAV9-HP-708-00002 | N709V/E712D |
| AAV9-HP-710-00002 | E712D/A714T |
| AAV9-HP-712-00002 | E712D/A714T/N716D |
| AAV9-HP-714-00002 | A714T/N716D/E718N |
| AAV9-HP-716-00002 | N716D/E718N |
| AAV9-HP-718-00002 | E718N |

¹The following system is used to name the hexapeptide scanning AAV9 mutants. The left three digits indicate the first amino acid position of the hexapeptide based on AAV9 VP1. The right five digits indicate AAV serotype from which each hexapeptide is derived: 10000, AAV1; 06000, AAV6; 00700, AAV7; 00080, AAV8; and 00009, AAV9; and 00002, AAV2. When a hexapeptide amino acid sequence is shared with multiple serotypes, the right five digits have more than one positive integer. AAV9-HP-584-00002 and AAV9-HP-586-00002 are poorly produced, and therefore, the data are not collected from these two mutants.

TABLE 2
Dodecapeptide AAV9-derived Mutants

| Name of mutant¹ | Amino acid substitutions |
|---|---|
| AAV9-DP-538-00002 | S540V/T548S/G549E |
| AAV9-DP-540-00002 | S540V/T548S/G549E/R550K/D551T |
| AAV9-DP-542-00002 | T548S/G549E/R550K/D551T |
| AAV9-DP-544-00002 | T548S/G549E/R550K/D551T/A555I |
| AAV9-DP-546-00002 | T548S/G549E/R550K/D551T/A555I/D556E |
| AAV9-DP-552-00002 | A555I/D556E/N562D |
| AAV9-DP-550-00002 | R550K/D551T/A555I/D556E |
| AAV9-DP-574-00002 | S576Q/Q579S/A581S/H584L |
| AAV9-DP-576-00002 | S576Q/Q579S/A581S/H584L/S586R/A587G |
| AAV9-DP-578-00002 | Q579S/A581S/H584L/S586R/A587G/Q588N/A589R |
| AAV9-DP-580-00002 | A581S/H584L/S586R/A587G/Q588N/A589R |
| AAV9-DP-582-00002 | H584L/S586R/A587G/Q588N/A589R/Q592A |
| AAV9-DP-584-00002 | H584L/S586R/A587G/Q588N/A589R/Q592A/G594A/W595D |
| AAV9-DP-586-00002 | S586R/A587G/Q588N/A589R/Q592A/G594A/W595D/Q597N |
| AAV9-DP-588-00002 | Q588N/A589R/Q592A/G594A/W595D/Q597N/N598T |
| AAV9-DP-590-00002 | Q592A/G594A/W595D/Q597N/N598T/I601V |
| AAV9-DP-704-00002 | N706Y/N709V/E712D/A714T |
| AAV9-DP-706-00002 | N706Y/N709V/E712D/A714T/N716D |
| AAV9-DP-708-00002 | N709V/E712D/A714T/N716D/E718N |
| AAV9-DP-710-00002 | E712D/A714T/N716D/E718N |
| AAV9-DP-714-00002 | A714T/N716D/E718N |

¹The same system as that for the hexapeptide scanning AAV9 mutants is used to name the dodecapeptide scanning AAV9 mutants. AAV9-DP-578-00002 and AAV9-DP-580-00002 are poorly produced, and therefore, the data are not collected from these two mutants.

TABLE 3
Dodecapeptide Scanning AAV5-derived Mutants

| Name of mutant¹ | Amino acid substitutions |
|---|---|
| AAV5DP-233-00002 | V238A/S241T |
| AAV5DP-235-00002 | V238A/S241T/Q246L |
| AAV5DP-237-00002 | V238A/S241T/Q246L/R248K |
| AAV5DP-239-00002 | S241T/Q246L/R248K/E249Q |
| AAV5DP-241-00002 | S241T/Q246L/R248K/E249Q/K251S |
| AAV5DP-243-00002 | Q246L/R248K/E249Q/K251S/G253Q |
| AAV5DP-245-00002 | Q246L/R248K/E249Q/K251S/G253Q/V255/D256G/G257A |
| AAV5DP-247-00002 | R248K/E249Q/K251S/G253Q/V255/D256G/G257A |
| AAV5DP-249-00002 | E249Q/K251S/G253Q/V255/D256G/G257A/A260D |
| AAV5DP-251-00002 | K251S/G253Q/V255/D256G/G257A/A260D/A262H |
| AAV5DP-253-00002 | G253Q/V255/D256G/G257A/A260D/A262H |
| AAV5DP-256-00002 | D256G/G257A/A260D/A262H |
| AAV5DP-258-00002 | A260D/A262H |
| AAV5DP-302-00002 | R303N/V304F/I306L |
| AAV5DP-304-00002 | V304F/I306L |
| AAV5DP-306-00002 | I306L/V316Q/Q317N |
| AAV5DP-308-00002 | V316Q/Q317N/S319G |
| AAV5DP-318-00002 | S319G |
| AAV5DP-326-00002 | D337S |
| AAV5DP-430-00002 | V431I/R437Y/F438L/V439S/S440R |
| AAV5DP-432-00002 | R437Y/F438L/V439S/S440R/N443T |
| AAV5DP-434-00002 | R437Y/F438L/V439S/S440R/N443T/T444P/G445S |
| AAV5DP-436-00002 | R437Y/F438L/V439S/S440R/N443T/T444P/G445S/V447T |
| AAV5DP-438-00002 | F438L/V439S/S440R/N443T/T444P/G445S/V447TTT |
| AAV5DP-440-00002 | S440R/N443T/T444P/G445S/V447TTTQS |
| AAV5DP-442-00002 | N443T/T444P/G445S/V447TTTQSRL |
| AAV5DP-444-00002 | T444P/G445S/V447TTTQSRL |
| AAV5DP-446-00002 | V447TTTQSRL/N450S/K451Q |
| AAV5DP-448a-00002 | Q448TTQSRLQ/N450S/K451Q/N452A/L453G |
| AAV5DP-448b-00002 | Q448QSRLQ/N450S/K451Q/N452A/L453G/G455S |
| AAV5DP-448c-00002 | Q448RLQ/N450S/K451Q/N452A/L453G/G455S/R456D/Y457I |
| AAV5DP-448d-00002 | N450S/K451Q/N452A/L453G/G455S/R456D/Y457I/A458R/N459D |
| AAV5DP-450-00002 | N450S/K451Q/N452A/L453G/G455S/R456D/Y457I/A458R/N459D/T460Q/Y461S |
| AAV5DP-452-00002 | N452A/L453G/G455S/R456D/Y457I/A458R/N459D/T460Q/Y461S/K462R |
| AAV5DP-454-00002 | G455S/R456D/Y457I/A458R/N459D/T460Q/Y461S/K462R/F465L |
| AAV5DP-561-00002 | Y563T/N564E/V565Q/G566Y/Q568S/M569V/A570S |
| AAV5DP-563-00002 | Y563T/N564E/V565Q/G566Y/Q568S/M569V/A570S/N573L |
| AAV5DP-565-00002 | V565Q/G566Y/Q568S/M569V/A570S/N573L/S575R/S576G |
| AAV5DP-567-00002 | Q568S/M569V/A570S/N573L/S575R/S576G/T577N/T578R |
| AAV5DP-569-00002 | M569V/A570S/N573L/S575R/S576G/T577N/T578R/A579Q/P580A |
| AAV5DP-571-00002 | N573L/S575R/S576G/T577N/T578R/A579Q/P580A |
| AAV5DP-573-00002 | N573L/S575R/S576G/T577N/T578R/A579Q/P580A/G583A/T584D |
| AAV5DP-575-00002 | S575R/S576G/T577N/T578R/A579Q/P580A/G583A/T584D/Y585V |
| AAV5DP-577-00002 | T577N/T578R/A579Q/P580A/G583A/T584D/Y585V/L587T |
| AAV5DP-579-00002 | A579Q/P580A/G583A/T584D/Y585V/L587T/E589G/I590V |
| AAV5DP-581-00002 | G583A/T584D/Y585V/L587T/E589G/I590V/V591L |
| AAV5DP-641-00002 | G645A/I647PS/S649T |
| AAV5DP-643-00002 | G645A/I647PS/S649T/D652A/V653A |
| AAV5DP-645-00002 | G645A/I647PS/S649T/D652A/V653A/P654K/V655F |
| AAV5DP-647-00002 | I647PS/S649T/D652A/V653A/P654K/V655F/S656A |
| AAV5DP-648-00002 | S649T/D652A/V653A/P654K/V655F/S656A |
| AAV5DP-650-00002 | D652A/V653A/P654K/V655F/S656A |
| AAV5DP-654-00002 | P654K/V655F/S656A |
| AAV5DP-656-00002 | S656A |
| AAV5DP-658-00002 | T668S |
| AAV5DP-688-00002 | N691S/D695K/P696S/Q697V/F698N |
| AAV5DP-692-00002 | D695K/P696S/Q697V/F698N/A702T/P703V |
| AAV5DP-694-00002 | D695K/P696S/Q697V/F698N/A702T/P703V/S705T |
| AAV5DP-696-00002 | P696S/Q697V/F698N/A702T/P703V/S705T/T706N |
| AAV5DP-698-00002 | F698N/A702T/P703V/S705T/T706N/E708V |
| AAV5DP-700-00002 | A702T/P703V/S705T/T706N/E708V/R710S/T711E |
| AAV5DP-702-00002 | A702T/P703V/S705T/T706N/E708V/R710S/T711E/T712P |
| AAV5DP-704-00002 | S705T/T706N/E708V/R710S/T711E/T712P |
| AAV5DP-706-00002 | T706N/E708V/R710S/T711E/T712P |
| AAV5DP-708-00002 | E708V/R710S/T711E/T712P |
| AAV5DP-710-00002 | R710S/T711E/T712P |
| AAV5DP-712-00002 | T712P/P723N |
| AAV5DP-714-00002 | P723N |

¹The same system as that for the hexapeptide scanning AAV9 mutants is used to name the dodecapeptide scanning AAV5 mutants. We have created and tested AAV vector production using these 68 capsids. The 18 mutants that were not included in the AAV5-DP libraries are those that do not produce sufficient titer for the downstream library preparation.

TABLE 4
AAV2Ep123 mutants

| Name of | | | |
|---|---|---|---|
| mutant | Epitope 1¹ | Epitope 2¹ | Epitope 3¹ |
| Wild type | TTQSRLQ (SEQ ID NO: 17) | AAKFA (SEQ ID NO: 6) | TVDTN (SEQ ID NO: 18) |
| Ep123mt1 | GGTAATE (SEQ ID NO: 14) | PARQL (SEQ ID NO: 15) | SVDGN (SEQ ID NO: 16) |
| Ep123mt2 | GGTAATE (SEQ ID NO: 14) | PRPVQ (SEQ ID NO: 19) | TVDTN (SEQ ID NO: 18) |
| Ep123mt3 | GGTAATE (SEQ ID NO: 14) | AAKFA (SEQ ID NO: 6) | TVDTN (SEQ ID NO: 18) |
| Ep123mt4 | TQEARPG (SEQ ID NO: 20) | PSALM (SEQ ID NO: 21) | TVDTN (SEQ ID NO: 18) |
| Ep123mt5 | TPTPQFS (SEQ ID NO: 22) | ADSLL (SEQ ID NO: 23) | TVDTN (SEQ ID NO: 18) |
| Ep123mt6 | TLEPLIT (SEQ ID NO: 24) | PASVM (SEQ ID NO: 25) | TVDTN (SEQ ID NO: 18) |
| Ep123mt7 | PFETDLM (SEQ ID NO: 26) | PRPLM (SEQ ID NO: 27) | TVDTN (SEQ ID NO: 18) |
| Ep123mt8 | LQEAHLT (SEQ ID NO: 28) | AAKFA (SEQ ID NO: 6) | TVDTN (SEQ ID NO: 18) |
| Ep123mt9 | EEGGRPK (SEQ ID NO: 29) | AQPVM (SEQ ID NO: 30) | TVDTN (SEQ ID NO: 18) |
| Ep123mt10 | EGDGGCL (SEQ ID NO: 31) | AAKFA (SEQ ID NO: 6) | TVDTN (SEQ ID NO: 18) |
| Ep123mt11 | DGGAGSW (SEQ ID NO: 32) | SEKQL (SEQ ID NO: 33) | TVDTN (SEQ ID NO: 18) |
| Ep123mt12 | AEGGGGG (SEQ ID NO: 34) | APAMC (SEQ ID NO: 35) | TVDTN (SEQ ID NO: 18) |
| Ep123mt13 | AGGGEMG (SEQ ID NO: 36) | SEKQL (SEQ ID NO: 33) | TVDTN (SEQ ID NO: 18) |
| Ep123mt14 | GEAAAPA (SEQ ID NO: 37) | AAKFA (SEQ ID NO: 6) | TVDTN (SEQ ID NO: 18) |
| Ep123mt15 | SVEGGAW (SEQ ID NO: 38) | DRRLL (SEQ ID NO: 39) | TVDTN (SEQ ID NO: 18) |
| Ep123mt16 | SLASTLE (SEQ ID NO: 40) | TLPMK (SEQ ID NO: 41) | TVDTN (SEQ ID NO: 18) |

¹The amino acid positions in the AAV2 capsid VP1 protein are 455-461, 663-667 and 713-717 for Epitopes 1, 2 and 3 respectively.

All references cited in this disclosure are incorporated by reference in their entirety.

It will be apparent to those having skill in the art that many changes may be made to the details of the above-described embodiments without departing from the underlying principles of the invention. The scope of the present invention should, therefore, be determined only by the following claims.

1-55. (canceled)

56. An AAV capsid mutant protein comprising at least one altered capsid epitope, wherein the at least one altered capsid epitope comprises at least one amino acid mutation in a position corresponding to AAV2 amino acid positions selected from the group consisting of 439-469, 650-672, 243-271, 320-337, 498-516, 523-533, 534-560, 570-596, 700-728, and 409-422.

57. The AAV capsid mutant protein of claim 56, wherein the AAV capsid mutant protein is configured to escape antibody binding or neutralization.

58. The AAV capsid mutant protein of claim 56, wherein the at least one altered capsid epitope comprises an alteration of at least one epitope selected from the group consisting of SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, and SEQ ID NO: 52.

59. The AAV capsid mutant protein of claim 58, wherein the at least one altered capsid epitope comprising an alteration of the epitope of SEQ ID NO: 5.

60. The AAV capsid mutant protein of claim 58, wherein the at least one altered capsid epitope comprising an alteration of the epitope of SEQ ID NO: 6.

61. The AAV capsid mutant protein of claim 58, wherein the at least one altered capsid epitope comprising an alteration of the epitope of SEQ ID NO: 7.

62. The AAV capsid mutant protein of claim 56, wherein the at least one altered capsid epitope comprises an alteration of at least one amino acid mutation relative to at least one epitope selected from the group consisting of SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, and SEQ ID NO: 52.

63. The AAV capsid mutant protein of claim 56, wherein the at least one altered capsid epitope comprises at least two amino acid mutations SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, and SEQ ID NO: 52.

64. The AAV capsid mutant protein of claim 56, wherein the at least one altered capsid epitope comprises at least three amino acid mutations SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, and SEQ ID NO: 52.

65. The AAV capsid mutant protein of claim 56, wherein the at least one altered capsid epitope comprises at least four amino acid mutations SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, and SEQ ID NO: 52.

66. The AAV capsid mutant protein of claim 66, wherein the AAV capsid mutant protein is derived from a wild-type AAV capsid protein selected from the group consisting of AAV1, AAV2, AAV3, AAV4, AAV5, AAV6, AAV7, AAV8, AAV9, AAV10, AAV11, AAV12, and AAV13.

67. The AAV capsid mutant protein of claim 66, wherein the wild-type AAV capsid protein is AAV2.

68. The AAV capsid mutant protein of claim 66, wherein the wild-type AAV capsid protein is AAV9.

69. A method of identifying one or more epitopes on an AAV capsid protein of a first AAV strain, the method comprising the steps of:
(a) preparing a plurality of AAV capsid mutants, wherein each AAV capsid mutant comprises an altered capsid protein from a second AAV strain, the altered capsid protein comprising one or more altered amino acids, and wherein the one or more altered amino acids are replaced by one or more of the corresponding amino acids from the AAV capsid protein of the first AAV strain;
(b) reacting the plurality of AAV capsid mutants with a plurality of antibodies, wherein each antibody binds to one or more epitopes on the AAV capsid protein of the first AAV strain;
(c) collecting the AAV capsid mutants that bind to one or more antibodies; and Identifying the AAV capsid mutants that bind to one or more antibodies.

70. The method of claim 69, wherein the first AAV strain is AAV2.

71. The method of claim 69, wherein the second AAV strain is AAV9.

72. The method of claim 69, wherein the second AAV strain is AAV5.

73. The method of claim 69, wherein collecting the AAV capsid mutants that bind to one or more antibodies comprises immunoprecipitating the AAV capsid mutants that bind to one or more antibodies.

74. The method of claim 69, wherein collecting the AAV capsid mutants that bind to one or more antibodies comprises determining pharmacokinetic profiles of mutants intravenously infused into an animal that bind to one or more antibodies present in the circulating blood in the animal.


SEQUENCE LISTING (65 sequences)

SEQ ID NO: 1 - protein, 6 aa, adeno-associated virus 1
QSGSAQ

SEQ ID NO: 2 - protein, 5 aa, adeno-associated virus 9
GSGQN

SEQ ID NO: 3 - protein, 6 aa, Mus musculus
SSQSGA

SEQ ID NO: 4 - protein, 6 aa, Mus musculus
PSGTTT

SEQ ID NO: 5 - protein, 10 aa, Homo sapiens
TTQSRLQFSQ

SEQ ID NO: 6 - protein, 5 aa, Homo sapiens
AAKFA

SEQ ID NO: 7 - protein, 13 aa, Homo sapiens
NKSVNVDFTVDTN

SEQ ID NO: 8 - protein, 12 aa, Homo sapiens
QYGSVSTNLQRG

SEQ ID NO: 9 - protein, 29 aa, adeno-associated virus 2
STRTWALPTYNNHLYKQISSQSGASNDNH

SEQ ID NO: 10 - protein, 18 aa, adeno-associated virus 2
VKEVTQNDGTTTIANNLT

SEQ ID NO: 11 - protein, 9 aa, Homo sapiens
NTPSGTTTQ

SEQ ID NO: 12 - protein, 9 aa, Homo sapiens
LQRGNRQAA

SEQ ID NO: 13 - protein, 17 aa, Homo sapiens
VLIFGKQGSEKTNVDIE

SEQ ID NO: 14 - protein, 7 aa, Artificial Sequence
  AAV2Ep123mt1
GGTAATE

SEQ ID NO: 15 - protein, 5 aa, Artificial Sequence
  AAV2Ep123mt1
PARQL

SEQ ID NO: 16 - protein, 5 aa, Artificial Sequence
  AAV2Ep123mt1
SVDGN

SEQ ID NO: 17 - protein, 7 aa, adeno-associated virus 2
TTQSRLQ

SEQ ID NO: 18 - protein, 5 aa, Artificial Sequence
  AAV2Ep123 mutant
TVDTN

SEQ ID NO: 19 - protein, 5 aa, Artificial Sequence
  AAV2Ep123 mutant
PRPVQ

SEQ ID NO: 20 - protein, 7 aa, Artificial Sequence
  AAV2Ep123 mutant
TQEARPG

SEQ ID NO: 21 - protein, 5 aa, Artificial Sequence
  AAV2Ep123 mutant
PSALM

SEQ ID NO: 22 - protein, 7 aa, Artificial Sequence
  AAV2Ep123 mutant
TPTPQFS

SEQ ID NO: 23 - protein, 5 aa, Artificial Sequence
  AAV2Ep123 mutant
ADSLL

SEQ ID NO: 24 - protein, 7 aa, Artificial Sequence
  AAV2Ep123 mutant
TLEPLIT

SEQ ID NO: 25 - protein, 5 aa, Artificial Sequence
  AAV2Ep123 mutant
PASVM

SEQ ID NO: 26 - protein, 7 aa, Artificial Sequence
  AAV2Ep123 mutant
PFETDLM

SEQ ID NO: 27 - protein, 5 aa, Artificial Sequence
  AAV2Ep123 mutant
PRPLM

SEQ ID NO: 28 - protein, 7 aa, Artificial Sequence
  AAV2Ep123 mutant
LQEAHLT

SEQ ID NO: 29 - protein, 7 aa, Artificial Sequence
  AAV2Ep123 mutant
EEGGRPK

SEQ ID NO: 30 - protein, 5 aa, Artificial Sequence
  AAV2Ep123 mutant
AQPVM

SEQ ID NO: 31 - protein, 7 aa, Artificial Sequence
  AAV2Ep123 mutant
EGDGGCL

SEQ ID NO: 32 - protein, 7 aa, Artificial Sequence
  AAV2Ep123 mutant
DGGAGSW

SEQ ID NO: 33 - protein, 5 aa, Artificial Sequence
  AAV2Ep123 mutant
SEKQL

SEQ ID NO: 34 - protein, 7 aa, Artificial Sequence
  AAV2Ep123 mutant
AEGGGGG

SEQ ID NO: 35 - protein, 5 aa, Artificial Sequence
  AAV2Ep123 mutant
APAMC

SEQ ID NO: 36 - protein, 7 aa, Artificial Sequence
  AAV2Ep123 mutant
AGGGEMG

SEQ ID NO: 37 - protein, 7 aa, Artificial Sequence
  AAV2Ep123 mutant
GEAAAPA

SEQ ID NO: 38 - protein, 7 aa, Artificial Sequence
  AAV2Ep123 mutant
SVEGGAW

SEQ ID NO: 39 - protein, 5 aa, Artificial Sequence
  AAV2Ep123 mutant
DRRLL

SEQ ID NO: 40 - protein, 7 aa, Artificial Sequence
  AAV2Ep123 mutant
SLASTLE

SEQ ID NO: 41 - protein, 5 aa, Artificial Sequence
  AAV2Ep123 mutant
TLPMK

SEQ ID NO: 42 - protein, 8 aa, adeno-associated virus 9
LGSAHEGC

SEQ ID NO: 43 - protein, 5 aa, adeno-associated virus 9
LTRNL

SEQ ID NO: 44 - protein, 8 aa, Artificial Sequence
  AAV9 mutant
AASAHEGC

SEQ ID NO: 45 - protein, 8 aa, Artificial Sequence
  AAV9 mutant
LGAAHEGC

SEQ ID NO: 46 - protein, 8 aa, Artificial Sequence
  AAV9 mutant
LGSAAAGC

SEQ ID NO: 47 - protein, 5 aa, Artificial Sequence
  AAV9 mutant
LTAAL

SEQ ID NO: 48 - protein, 5 aa, Artificial Sequence
  AAV9 mutant
LTRNA

SEQ ID NO: 49 - protein, 15 aa, adeno-associated virus 2
YLYYLSRTNTPSGTT

SEQ ID NO: 50 - protein, 9 aa, adeno-associated virus 2
RTNTPSGTT

SEQ ID NO: 51 - protein, 7 aa, adeno-associated virus 2
NTPSGTT

SEQ ID NO: 52 - protein, 14 aa, adeno-associated virus 2
FTFSYTFEDVPFHS

SEQ ID NO: 53 - protein, 735 aa, adeno-associated virus 2 [sequence omitted]

SEQ ID NO: 54 - protein, 736 aa, adeno-associated virus 1
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 55 - protein, 736 aa, adeno-associated virus 3B
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 56 - protein, 734 aa, adeno-associated virus 4
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 57 - protein, 724 aa, adeno-associated virus 5
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 58 - protein, 736 aa, adeno-associated virus 6
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 59 - protein, 737 aa, adeno-associated virus 7
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 60 - protein, 738 aa, adeno-associated virus 8
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 61 - protein, 736 aa, adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 62 - protein, 738 aa, adeno-associated virus 10
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGESESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQANTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 63 - protein, 733 aa, adeno-associated virus 11
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPLESPQEPDSSSGIGKKGKQPARKRLNFEEDTGAGDGPPEGSDTSAMSSDIEMRAAPGGNAVDAGQGSDGVGNASGDWHCDSTWSEGKVTTTSTRTWVLPTYNNHLYLRLGTTSSSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFEMAYNFEKVPFHSMYAHSQSLDRLMNPLLDQYLWHLQSTTSGETLNQGNAATTFGKIRSGDFAFYRKNWLPGPCVKQQRFSKTASQNYKIPASGGNALLKYDTHYTLNNRWSNIAPGPPMATAGPSDGDFSNAQLIFPGPSVTGNTTTSANNLLFTSEEEIAATNPRDTDMFGQIADNNQNATTAPITGNVTAMGVLPGMVWQNRDIYYQGPIWAKIPHADGHFHPSPLIGGFGLKHPPPQIFIKNTPVPANPATTFTAARVDSFITQYSTGQVAVQIEWEIEKERSKRWNPEVQFTSNYGNQSSMLWAPDTTGKYTEPRVIGSRYLTNHL

SEQ ID NO: 64 - protein, 742 aa, adeno-associated virus 12
MAADGYLPDWLEDNLSEGIREWWALKPGAPQPKANQQHQDNGRGLVLPGYKYLGPFNGLDKGEPVNEADAAALEHDKAYDKQLEQGDNPYLKYNHADAEFQQRLATDTSFGGNLGRAVFQAKKRILEPLGLVEEGVKTAPGKKRPLEKTPNRPTNPDSGKAPAKKKQKDGEPADSARRTLDFEDSGAGDGPPEGSSSGEMSHDAEMRAAPGGNAVEAGQGADGVGNASGDWHCDSTWSEGRVTTTSTRTWVLPTYNNHLYLRIGTTANSNTYNGFSTPWGYFDFNRFHCHFSPRDWQRLINNNWGLRPKSMRVKIFNIQVKEVTTSNGETTVANNLTSTVQIFADSTYELPYVMDAGQEGSFPPFPNDVFMVPQYGYCGVVTGKNQNQTDRNAFYCLEYFPSQMLRTGNNFEVSYQFEKVPFHSMYAHSQSLDRMMNPLLDQYLWHLQSTTTGNSLNQGTATTTYGKITTGDFAYYRKNWLPGACIKQQKFSKNANQNYKIPASGGDALLKYDTHTTLNGRWSNMAPGPPMATAGAGDSDFSNSQLIFAGPNPSGNTTTSSNNLLFTSEEEIATTNPRDTDMFGQIADNNQNATTAPHIANLDAMGIVPGMVWQNRDIYYQGPIWAKVPHTDGHFHPSPLMGGFGLKHPPPQIFIKNTPVPANPNTTFSAARINSFLTQYSTGQVAVQIDWEIQKEHSKRWNPEVQFTSNYGTQNSMLWAPDNAGNYHELRAIGSRFLTHHL

SEQ ID NO: 65 - protein, 733 aa, adeno-associated virus 13
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQERLQEDTSFGGNLGRAVFQAKKRILEPLGLVEEAAKTAPGKKRPVEQSPAEPDSSSGIGKSGQQPARKRLNFGQTGDTESVPDPQPLGQPPAAPSGVGSTTMASGGGAPMADNNEGADGVGNSSGNWHCDSQWLGDRVITTSTRTWALPTYNNHLYKQISSQSGATNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQTASGTQQSRLLFSQAGPTSMSLQAKNWLPGPCYRQQRLSKQANDNNNSNFPWTGATKYHLNGRDSLVNPGPAMASHKDDKEKFFPMHGTLIFGKEGTNANNADLENVMITDEEEIRTTNPVATEQYGTVSNNLQNSNAGPTTGTVNHQGALPGMVWQDRDVYLQGPIWAKIPHTDGHFHPSPLMGGFGLKHPPPQIMIKNTPVPANPPTNFSAAKFASFITQYSTGQVSVEIEWELQKENSKRWNPEIQYTSNYNKSVNVDFTVDTNGVYSEPRPIGTRYLTRNL